(19) United States
(12) Patent Application Publication
(10) Pub. No.: US 2026/0108617 A1
(43) Pub. Date: Apr. 23, 2026

(54) AUTO-TUNING DRUG REGULATOR CONSTRUCTIONS

(71) Applicant: ReThink64 Bionetworks PBC, Fremont, CA (US)

(72) Inventors: Tapash Jay SARKAR, Tomball, TX (US); Edward NJOO, Fremont, CA (US); Lorelei XIA, Fremont, CA (US); Akira YAMAMOTO, San Carlos, CA (US); Zachary BASHKIN, Fremont, CA (US); Warren CHANG, Fremont, CA (US)

(21) Appl. No.: 19/148,655

(22) PCT Filed: Jan. 17, 2024
(86) PCT No.: PCT/US2024/011827

§ 371 Date: Jul. 16, 2025

Related U.S. Application Data
(60) Provisional application No. 63/480,129, filed on Jan. 17, 2023.

(51) Int. Cl. A61K 47/65

(52) U.S. Cl. CPC A61K 47/65

(57) ABSTRACT

The present invention provides a regulator construct in which the drug efficacy is auto-regulated by the presence of a indicator enzyme. The construct is a chemical structure comprising: (a) a biorecognition component comprising a substrate recognized by an enzyme, (b) an incapacitation component, and (c) an active drug component. The incapacitation component provides a chemical incapacitation mechanism to render benign or incapacitate the drug component, after the substrate-enzyme interaction, allowing additionally for the creation of altered benign chemical structures while, otherwise, when said enzymes is inactive or absent the drug will take effect.

### CROSS-REFERENCE

This application claims the benefit of U.S. Provisional Application Ser. No. 63/480,129, filed Jan. 17, 2023, which is hereby incorporated by reference in its entirety.

### TECHNICAL FIELD

The present disclosure relates to regulator constructs for drug components, where the drug efficacy is regulated by the presence or activity of a selected indicator enzyme.

### BACKGROUND

For optimizing the efficacy of treatment, it is important to tune an intervention to the patient's specific condition. At the most rudimentary level, this is done through an initial or periodic screening for measured disease symptoms followed by physician prescription of the standardized dosing regimen appropriate for the stage of the disease. This standardized dose is typically established through trial-and-error during clinical treatments and achieves some criterion of average efficacy across a population. However, this stage-wise averaging of disease progression and treatment glosses over much of the pathological heterogeneity that exists between patients and, even the day to day variation, within a patient. This can lead to either undershooting the target efficacy or overdosing with resulting side effects. As such, there has been a push for more “personalized medicine” solutions. This entails the measurement of one or more biomarkers and accordingly adjustments in the dosing for that specific patient by the drug administrator or by the patients themselves. However, for meaningful improvement above the standard dosing paradigm, timely repeatable methodologies are needed based on measurement-dose correction. In most cases, this is impractical if not unrealizable and can potentially lead to mismanagement or misalignment of dose due to the repeated risk of time and measurement errors. Thus a more practical solution is greatly needed.

### SUMMARY

In some aspects, is a chemical structure comprising: (a) a biorecognition component comprising a substrate recognized by an enzyme, (b) an incapacitation component, covalently linked to the biorecognition component, and (c) a drug component covalently linked to the incapacitation component; wherein the incapacitation component provides an incapacitation mechanism to incapacitate the drug component upon substrate recognition and reaction with the enzyme; and wherein the drug is capable to execute its biological effect, but incapacitated after the recognition and reaction.

In some aspects, is an auto-regulating compound comprising: (a) a biorecognition component comprising a substrate that is recognized by an enzyme; (b) an incapacitation component; and (c) a drug component comprising a therapeutic agent, wherein the drug component is active in the auto-regulating compound; wherein: the incapacitation component is covalently linked to the biorecognition component and the drug component; and wherein when the enzyme reacts with the biorecognition component, a covalent bond tethering the biorecognition component is cleaved, triggering an incapacitation mechanism to incapacitate the drug component.

In some aspects, is an auto-regulating compound comprising: (a) a biorecognition component comprising a substrate that is recognized by an enzyme; (b) an incapacitation component; and (c) a drug component comprising a therapeutic agent, wherein the drug component retains its biological functional capacity in the auto-regulating compound; wherein: the incapacitation component is covalently linked to the biorecognition component and the drug component; and wherein when the biorecognition component binds to the enzyme, the biorecognition component is released from the incapacitation component, and triggers an incapacitation mechanism, wherein the incapacitation mechanism is the diversion, out-competition, breakdown, clearance, or mobility impairment of the auto-regulating compound, resulting in the incapacitation of the drug component.

In some embodiments, the chemical structure or the auto-regulating compound has a structure of A-B-C, C-A-B, C-A-B-C′, wherein A is the biorecognition component, B is the incapacitation component, C is the drug component, and C′ is an additional drug component. In some embodiments, the enzyme cleaves or alters a covalent bond tethering the biorecognition component to engage the incapacitation mechanism. In some embodiments, the enzyme cleaves the covalent bond. In some embodiments, the covalent bond is an amide bond.

In some embodiments, the enzyme is a hydrolase, an oxidoreductase, a transferase, a translocase, a lyase, a ligase, or an isomerase. In some embodiments, the hydrolase is a protease, a nuclease, a lipase, a phosphatase, or an esterase. In some embodiments, the protease is prostasin, matriptase, or CYLD lysin 63 deubiquitinase. In some embodiments, the nuclease is an endonuclease, an exonuclease, a DNase, a RNase, a topoisomerase, a recombinase, a ribozyme, or a RNA splicing enzyme. In some embodiments, the lipase is bile salt-dependent lipase, pancreatic lipase, lysosomal lipase, hepatic lipase, lipoprotein lipase, hormone-sensitive lipase, gastric lipase, endothelial lipase, or lingual lipase. In some embodiments, the phosphatase is phosphatidylinositol-3,4,5-trisphosphate 3-phosphatase, phosphatidic acid phosphatase type 2B, or inositol polyphosphate-5-phosphatase. In some embodiments, the esterase is acetylesterase or phosphodiesterase 2. In some embodiments, the enzyme alters the covalent bond between the biorecognition component and the incapacitation component by transfer of electrons or bonds from one substrate to another. In some embodiments, the enzyme is oxidoreductase or transferase. In some embodiments, wherein the oxidoreductase is aldehyde dehydrogenase 2. In some embodiments, the transferase is methyltransferase or 3-hydroxy-3-methylglutaryl-CoA synthase 2. In some embodiments, the enzyme is located in a subject's extracellular space, cell membrane, cytoplasm, nucleus, or nuclear membrane.

In some embodiments, the substrate comprises a peptide sequence or a biorecognition element, wherein the enzyme recognizes the peptide sequence or the biorecognition element. In some embodiments, the peptide sequence is recognized by protease. In some embodiments, the peptide sequence is QAR. In some embodiments, the biorecognition element is an oligonucleotide, lipid or lipidic ester, or phosphate groups.

In some embodiments, the incapacitation component is a set of linkers that comprises a release trigger covalently linked to the biorecognition element, wherein the release trigger is cleaved from the biorecognition element upon substrate biorecognition and reaction with the enzyme. In some embodiments, the release trigger comprises a 2- or 4-substituted benzyl carbamate. In some embodiments, the linkers degrade into biologically inert components upon substrate biorecognition and reaction with the enzyme.

In some embodiments, the incapacitation component comprises a linker of Formula (I):

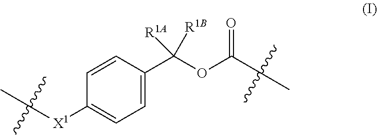

wherein: X¹ is O or NH; and R^{1A} and R^{1B} are each independently H or C₁₋₆ alkyl.

In some embodiments, the incapacitation component comprises a linker of Formula (I-A):

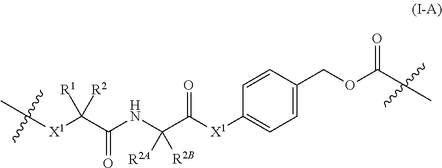

wherein: X¹ is O or NH; X² is O or NH; and R^{1A}, R^{1B}, R^{2A}, and R^{2B} are each independently H or C₁₋₆ alkyl.

In some embodiments, the incapacitation mechanism is decoy presentation, competitive disabling, drug component dismemberment, increased clearability, or mobility reduction as described herein. In some embodiments, the incapacitation mechanism further comprising a cell entry component.

In some embodiments, the drug component retains efficacy when the incapacitation mechanism is not triggered, wherein the incapacitation component is covalently bonded to the biorecognition component. In some embodiments, wherein the drug component is a small molecule, macromolecule, peptide, protein, nucleic acid, lipid, metabolite, antibody, or hormone. In some embodiments, the drug component is an alkylating agent. In some embodiments, the alkylating agent is an alkyl chloride, vinyl sulfone, acrylate, or epoxide. In some embodiments, the alkylating agent is α-chloracetamide, α-halocarbonyl, acrylate, vinylsulfone, epoxycarbonyl, diazirine, diazo, glufosfamide, ifosfomide, or lomustine. In some embodiments, the drug component is an intercalating agent. In some embodiments, the intercalating agent is amonafide. In some embodiments, the drug component is an antimetabolite. In some embodiments, the antimetabolite is floxuridine, gemcitabine, or 5-fluorouracil.

In some embodiments, the drug component is a small molecule enzyme inhibitor. In some embodiments, the small molecule enzyme inhibitor inhibits TET1, TET2, or a combination thereof. In some embodiments, the small molecule enzyme inhibitor is Bobcat 339. In some embodiments, the small molecule enzyme inhibitor inhibits topoisomerase. In some embodiments, the small molecule enzyme inhibitor is amonafide, SN-38, or etoposide. In some embodiments, the drug component is a small molecule bromodomain inhibitor. In some embodiments, the small molecule bromodomain inhibitor is a BET inhibitor. In some embodiments, the small molecule bromodomain inhibitor is JQ-1. In some embodiments, the drug component is a small molecule kinase inhibitor. In some embodiments, the small molecule kinase inhibitor inhibits mTOR. In some embodiments, the small molecule kinase inhibitor is rapamycin. In some embodiments, the drug component comprises at least two subcomponents that have different biological targets. In some embodiments, the drug component is PROTAC. In some embodiments, PROTAC comprises a BRD4 ligand. In some embodiments, BRD4 ligand is JQ-1. In some embodiments, PROTAC comprises a TrkA ligand. In some embodiments, the TrkA ligand has the peptide sequence IENPQYFSDA.

In some embodiments, the drug component is a proteasome inhibitor. In some embodiments, the proteasome inhibitor is bortezomib, ixazomib, or MG132. In some embodiments, the drug component is a proteasome activator. In some embodiments, the proteasome activator is a peptide. In some embodiments, the drug component is an imaging agent. In some embodiments, the imaging agent is fluorescein.

In some embodiments, the compound is a structure of Formula (II):

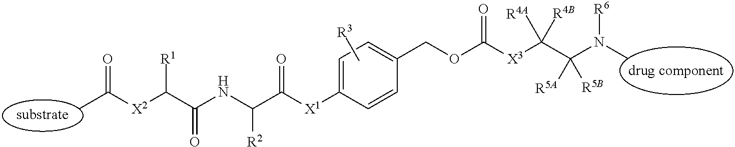

wherein: X¹, X², and X³ are each independently O, NH, or S; R¹, R², R³, R^{4A}, R^{4B}, R^{5A}, R^{5B}, and R⁶ are each independently H or C₁₋₆ alkyl; substrate is a peptide; and drug component is a therapeutic agent. In some embodiments, the compound of Formula (II) is as described herein.

In some embodiments, compound is:

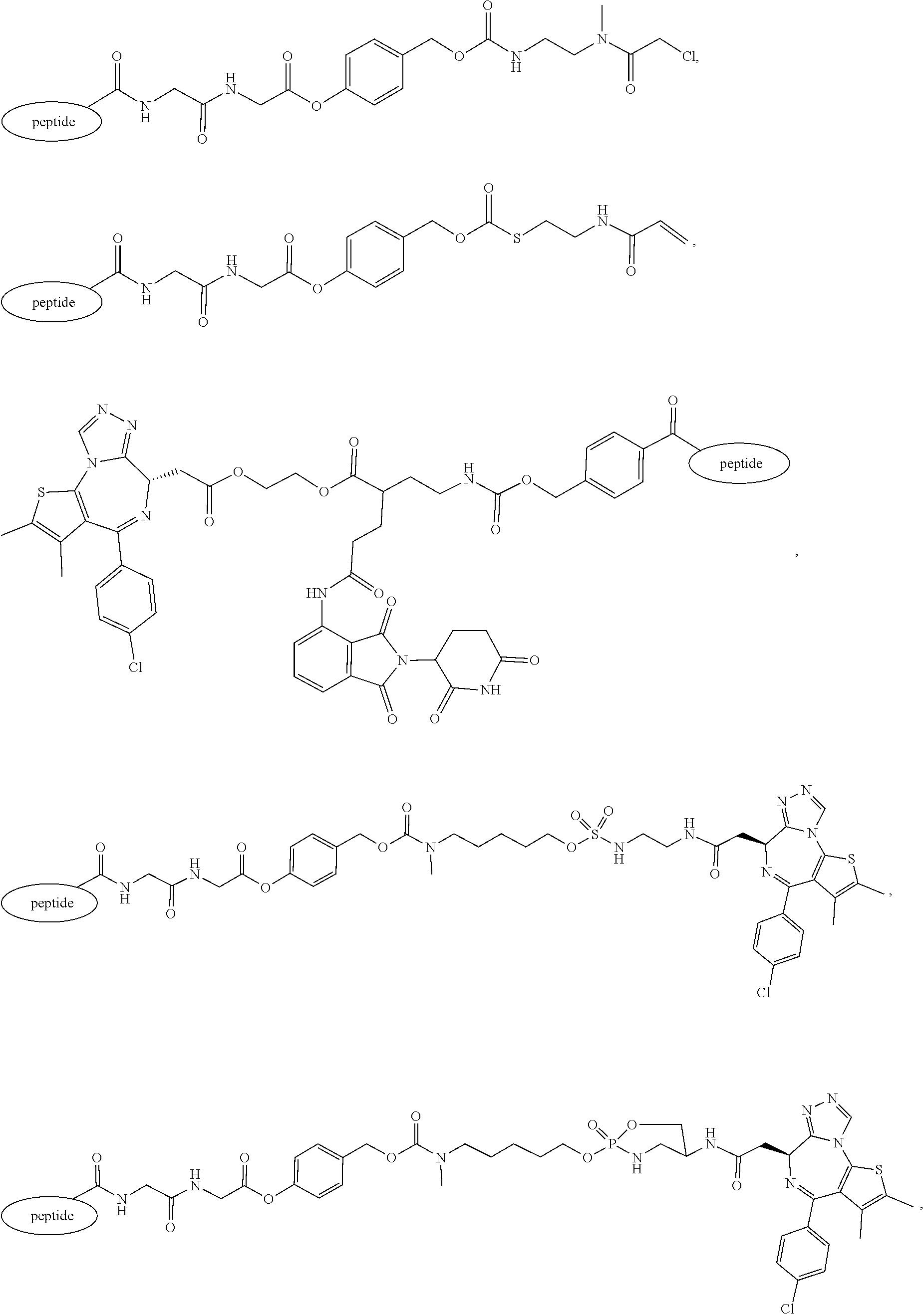

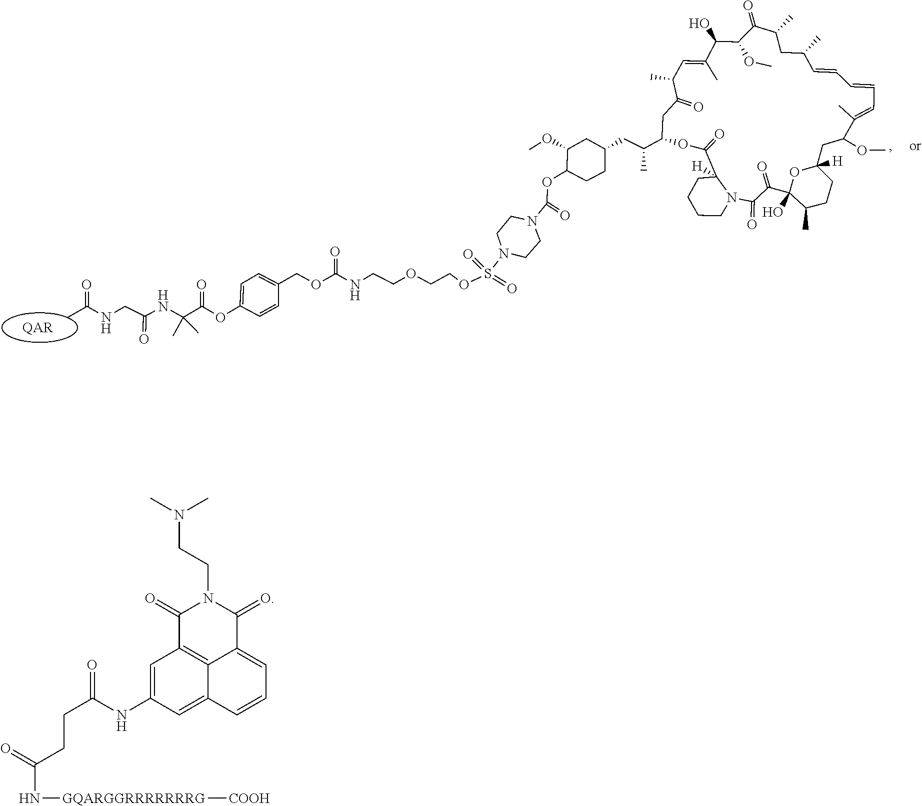

In some embodiments, is a method of treating a disease or disorder, the method comprising administering to the subject, an auto-regulating compound described herein, or a pharmaceutically acceptable salt or solvate thereof.

In some aspects, is a method of tuning the amount of a therapeutic agent for the treatment of a disease or disorder in a subject, wherein the subject is administered a therapeutically effective amount of the therapeutic agent when the disease or disorder is present or progressing, and the subject is not administered a therapeutically effect amount of the therapeutic agent when the disease or disorder is mitigated or in areas where there are healthy cells, tissues, or organs, the method comprising: administering to the subject an auto-regulating compound, wherein the auto-regulating compound comprises (i) a biorecognition component comprising a substrate; (ii) an incapacitation component; and (iii) a drug component comprising the therapeutic agent, wherein the incapacitation component is covalently linked to the biorecognition component and the drug component; wherein the auto-regulating compound has an incapacitation mechanism, wherein when the disease or disorder is progressing, the auto-regulation compound carries out its biological function; wherein when the disease or disorder is mitigated or where there are healthy cells, tissues, or organs, the auto-regulation compound is incapacitated via an incapacitation mechanism comprising: (a) reacting the biorecognition component with an enzyme, wherein the enzyme is absent or in low concentration during and in regions of disease or disorder presence or progression, and the enzyme is in higher concentration during disease or disorder mitigation or where there are healthy cells, tissues, or organs; (b) cleaving of a covalent bond between the biorecognition component and the incapacitation component, thereby triggering the incapacitation mechanism; and (c) incapacitating the drug component with the incapacitation component which is no longer covalently linked to the biorecognition component;
thereby tuning the amount of the therapeutic agent for the treatment of a disease or disorder, and reducing side and off target effects.

In some embodiments, the disease or disorder has a target enzyme which (1) decreases in presence or activity as the disease progresses, and (2) has a specific substrate recognized by the target enzyme. In some embodiments, the disease or disorder is cancer. In some embodiments, the cancer has reduction of prostasin enzyme. In some embodiments, wherein the cancer is breast cancer, colorectal cancer, squamous cell carcinoma, or prostate cancer. In some embodiments, wherein the disease or disorder is protein aggregation disease. In some embodiments, the disease is a neurodegenerative disease, Alzheimer's, Parkinson's, amyotrophic lateral sclerosis, dementia with Lewy bodies, frontotemporal dementia, or Huntington's disease.

In some aspects is a kit for tuning the amount of a therapeutic agent for the treatment of a disease or disorder, the kit comprising: (a) a pharmaceutical composition comprising the auto-regulating compound described herein and a pharmaceutically acceptable excipient; and (b) an instruction manual for the usage of the auto-regulating compound.

### BRIEF DESCRIPTION OF THE DRAWINGS

FIG. 1 shows simple binary illustration of mechanisms of autoregulation through negative feedback. Purpose of the drug should be to restore healthy phenotype reflected by biological indicators. Examples would be drugs that support healthy functions/cells or drugs that eliminate unhealthy functions/cells. Mechanism need not be purely on or off, but can be gradual incapacitation that is inversely proportional to the biological indicator level.

FIG. 2 shows qPCR for Prostasin Gene Expression (normalized to normal cell line) indicating diminished level of prostasin shown in multiple breast cancer cell lines as to breast epithelial cell lines.

FIG. 3 shows the release trigger architecture undergoes a 1,6 (O,N)-acyl shift once the biorecognition element is recognized and cleaved. This causes a 1,6-methide elimination which can be used to trigger a number of downstream reactions for the various modalities of incapacitation mechanism. This element is diversifiable and rapidly implements the logic of the adaptive drug.

FIG. 4 shows the specific implementation of the decoy presentation modality for an adaptive alkylating agent, the release trigger architecture is used to expose a primary amine. The electrophilic alkylating agent will then react with the amine in an SN2-type cyclization. This intramolecular reaction is highly favored over the intermolecular reaction of the alkylating agent with DNA and yields inert by products, incapacitating the drug's function as a genotoxin. The “warhead” of the alkylating agent is diversifiable—in an example can be chlorine that converts to free a chloride ion.

FIG. 5 shows synthesis route for adaptive alkylating agent using release trigger, peptide for biorecognition element and alkyl chloride drug active. Intermediaries are numbered below each compound.

FIG. 6 shows early stages of the adaptive alkylating agent synthesis begin with commercially available 4-hydroxybenzyl alcohol treated with tert-butyl dimethyl silyl chloride, imidazole, and 4-N,N-dimethylaminopyridine (DMAP) which affected selective monosilylation to afford hydroxybenzyl silyl ether intermediary 2. Subsequent Stegleich esterification of intermediary 2 with FMOC-protected glycine followed by desilylative acidic workup gave FMOC-protected hydroxyester intermediary 3. Depicted are characterizations of key intermediaries.

FIG. 7 shows middle stages of the adaptive alkylating agent synthesis involve the addition of phenyl chloroformate for efficient conversion of alcohol intermediary 3 to the asymmetric carbonate intermediary 4. Then upon addition of mono-(tert-butoxycarbony) protected N-methylethylenediamine, converts intermediary 4 to a Boc-protected carbamate intermediary 5. Removal of the Boc group was found to be efficiently conducted in a 25% v/v solution of trifluoroacetic acid (TFA) in any light chlorinated solvent (e.g. dichloroethane, DCE; chloroform, CHCl₃; dichloromethane, CH₂Cl₂) to afford the amine intermediary 6, which is chromatographically purified. Depicted are characterizations of key intermediaries.

FIG. 8 shows final stages of the adaptive alkylating agent synthesis involve acylation of the amine intermediary 6 with any α-haloacetyl group with reagents such as 2-chloroacetyl chloride to afford the haloacetamide intermediary 7, which is envisioned to be a key diversifiable intermediate. Treatment of intermediary 7 with 4-methylpiperidine in acetonitrile gave efficient removal of the FMOC protecting group, but the resulting primary amine intermediary 8 was too polar to purify. This could be immediately acylated with any C-terminal acid of a peptide under standard amide coupling conditions including but not limited to HBTU/HOBt, HATU/HOBt, PyBOP/HOBt, which after deprotection, yields the final product, depicted is LC/MS plot.

FIG. 9 shows incubation of cDNA with adaptive alkylating agent shows 98% reduction in cDNA amplification of housekeeping gene (GAPDH) by qPCR. Recombination prostasin enzyme can be added to tune back up to 26% of normal amplification in this example. Enzyme alone introduces some noise but does not significantly change the amplification level. This is the analogue to synthesis and replication inhibition of DNA in vitro/vivo by the adaptive alkylating compound.

FIG. 10 shows the adaptive alkylating agent is incubated with recombinant prostasin enzyme and is analyzed by LC/MS to show the 1-methylpiperazine-one byproduct of the decoy interaction. This is not observed in absence of the enzyme incubation.

FIG. 11 shows the application of adaptive alkylating agent shows reduction in cell numbers for breast cancer lines of different subtypes (MCF7 and MDA-MB-231) without significant change in normal cell line (MCF10a).

FIG. 12 shows application of adaptive alkylating agent shows effect of varying applied dose (1×, half concentration and a quarter concentration as well as a no dose control C) on cancer line cell number reduction at 1 hr.

FIG. 13 shows application of adaptive alkylating agent shows effect of varying applied dose (1×, half concentration and a quarter concentration as well as a no dose control C) on cancer line cell number reduction at 2 hrs.

FIG. 14 shows application of adaptive alkylating agent shows effect of varying applied dose (1×, half concentration and a quarter concentration as well as a no dose control C) on cancer line cell number reduction at 24 hrs.

FIG. 15 shows mechanism for adaptive alkylating agent using biorecognition trigger, peptide for biorecognition element and alternative (acrylate) drug active.

FIG. 16 shows synthesis route for adaptive alkylating agent using biorecognition trigger, peptide for biorecognition element and alternative (acrylate) drug active. Intermediaries are numbered below each compound.

FIG. 17 shows a generalizable route with other drug actives and additional features. X is any 4-heteroatom substituted or aryl-substituted benzyl alcohol, where ‘heteroatom’ describes any nonmetallic element other than carbon or hydrogen, including nitrogen, oxygen (as in the present embodiment), sulfur, selenium, phosphorous, or other elements not included in this list, and where ‘aryl-substituted’, denoted by R1, indicates any mono-, di-, tri-, or tetrasubstituted ortho- or para-benzyl alcohol, wherein ‘substituted’ includes any linear or branched alkyl, alkenyl, alkynyl, aryl, heteroalkyl, alkenyl, alkynyl, heteroaryl, or halogen substitution on the aromatic ring of the starting material or any combinations, salts, or permutations thereof. The Fmoc-Glycine acid coupling partner in step b could be substituted with any FMOC or similarly protected amino acid, where R2 is any linear or branched alkyl, alkenyl, alkynyl, aryl, heteroalkyl, alkenyl, alkynyl, heteroaryl, or halogen substitution or any combinations, salts, or permutations thereof with any integer number of atoms from 1-100, including any stereoisomers, constitutional isomers, geometric isomers, or any combination thereof. In yet another example embodiment, reaction step c could consist of any 1,1-disubstituted carbonyl such as phosgene, triphosgene, diphosgene, 4-nitrophenyl chloroformate, 2,2,2-trichloroethyl chloroformate, carbonyldiimidazole, phenylchloroformate (as in the present embodiment) or any similar reagent which fit the aforementioned descriptor, including any 1,1-disubstituted thiocarbonyl equivalent reagent. In yet another example embodiment, the monoprotected diamine or hydroxyamine in coupling step d could be monoprotected with a Boc, Fmoc, Alloc, Troc, Cbz, or other similar protecting groups, and where R3, R4, R5, and R6 are any linear or branched alkyl, alkenyl, alkynyl, aryl, heteroalkyl, heteroaryl, or halogen substitution or any combinations, salts, or permutations thereof with any integer number of atoms from 1-100, including any stereoisomers, constitutional isomers, geometric isomers, or any combination thereof. In yet another example embodiment, the chloroacetyl moiety in step f might be substituted for any other alkylatable or acylatable or otherwise covalently modifiable drug component Y, including acrylates, diazo, α-haloacetates, α-halosulfonates, epoxypropanoates and the corresponding thioepoxide, aziridine, oxaziridine, or any analogous three or four membered strained system, including any alkyl, alkenyl, alkynyl, branched alkyl, alkenyl, alkynyl, heteroalkyl variation thereof. The C-carboxylic acid peptide in coupling step h may be substituted with any polypeptide or polypeptidomimentic or any polymeric or monomeric set of amino acids in a linear or branched moiety of any length, including side chains containing both natural and/or unnatural/synthetic amino acid side chains, including any stereoisomers or combinations or permutations or protected variations thereof, where ‘protected’ includes any common or non-common protecting group moiety, including but not limited to trityl ethers, t-butyl ethers, silyl ethers, Boc, Fmoc, Cbz, Troc, Alloc, or other similar or dissimilar protecting groups, including any N-alkylated or N-acylated amide backbones of said peptides, and where ‘amino acids’ includes α, β, γ, δ-amino acids or any other linear or branched alkyl or heteroalkyl length separating an amine and an acid moiety, including any linear, cyclic, polycyclic, or macrocyclic variations thereof, and including any salts, ion pairs, formulations, or synthetically modified variations thereof.

FIG. 18 shows synthesis route for adaptive alkylating agent using alternate biorecognition trigger that is simpler to synthesize. Compound still uses peptide for biorecognition element and alkyl chloride drug active. Intermediaries are numbered below each compound.

FIG. 19 shows characterization data. For the first step for the alternate release trigger, to a 100 mL oven dried round bottom flask fitted with a Teflon Stir Bar under nitrogen is added 4-hydroxybenzyl alcohol (5.10 g, 41 mmol, 1.0 equiv) in DMF (24 mL) was added imidazole (7.0 g, 103 mmol, 2.5 equiv). The reaction vessel is cooled to 0° C. in an ice bath and TBS-Cl (9.9 g, 65.6 mmol, 1.6 equiv) as a single portion. The reaction vessel is allowed to warm up to room temperature over 5 minutes, and the progress of the reaction is followed by thin layer chromatography (TLC, 1:1 EtOAc/hex.). Full conversion of the alcohol is achieved in 20 minutes, at which point the reaction mixture is quenched by the addition of 100.0 mL cold saturated ammonium chloride solution. The product is extracted in three portions of diethyl ether (3×150 mL) and the combined organic layers are back-extracted with 100 mL additional brine followed by 100 mL saturated lithium chloride to remove residual DMF. The resulting organic layers are combined, dried over anhydrous magnesium sulfate, and concentrated in vacuo. The crude product mixture is purified by silica gel flash chromatography (6.0 cm×18.0 cm) to afford silylphenol intermediary 2 as a viscous yellow oil. Given are characterizations of the intermediary 2.

FIG. 20 shows characterization data. For the second step for the alternate release trigger, to a 250 mL round bottom flask under nitrogen charged with a Teflon coated stir bar is added intermediary 2 (2.35 g, 10 mmol, 1 equiv), Boc-dimethylglycine (2.0 g, 10 mmol, 1 equiv.), EDC (2.95 g, 20 mmol, 2 equiv), DMAP (2.32 g, 20 mmol, 2 equiv) in dichloromethane (60 mL). The reaction is allowed to stir for 18 hr., at which point TLC analysis indicates complete conversion of starting materials to an ester product. Upon completion of the reaction, the mixture is worked up with the addition of ethyl acetate (100 mL) and brine (100 mL). The ester product is extracted in three portions of 100 mL of ethyl acetate and 100 mL brine. The combined organic layers are dried over magnesium sulfate, filtered, and concentrated in vacuo. The crude product is directly re-dissolved in a 4:1 mixture of acetonitrile and water (32 mL MeCN, 8 mL water). Formic acid (9.6 mL, 20% v/v) is added as a single portion. The reaction is stirred for 30 minutes at room temperature, at which point TLC analysis indicates full conversion of silyl ether product to alcohol intermediary 3. Upon completion, the reaction is quenched with the addition of 0.1 M sodium carbonate solution (100 mL) and the product is extracted in three portions of 100 mL of ethyl acetate. The combined organic layers are dried over magnesium sulfate, filtered, and concentrated in vacuo. The crude product is purified by silica gel flash chromatography (gradient of 10% 60% EtOAc/hex.; 6 cm×14 cm silica column) to afford intermediary 3 as a low-melting solid. Depicted are characterizations of the intermediary 3.

FIG. 21 shows characterization data. For the third step for the alternate release trigger, to a 50 mL oven dried round bottom flask fitted with a Teflon stir bar is added the alcohol intermediary 3 (505 mg, 1.67 mmol, 1 equiv), CDI (415 mg, 1.2 equiv). After 30 minutes, complete conversion of intermediary 3 to imidazole carbamate is observed by thin layer chromatography (TLC) analysis (R_{f}=0.4, 3:2 EtOAc/hex., pink stain in ninhydrin). After this, 0.25 mL N-methylimidazole in an additional 5 mL acetonitrile was added, followed by 216 μL ethylenediamine (3.33 mmol, 2 equiv). Complete conversion of imidazole carbamate to an amine is observed (R_{f}=0.2, 1:4 MeOH/DCM, purple stain in ninhydrin). At this point, the reaction is cooled on an ice bath and chloroacetyl chloride (0.392 mL, 5.0 mmol, 3 equiv) was added, along with 0.386 mL NMI (5.0 mmol, 3 equiv). After 5 minutes the ice bath was removed, and the reaction is allowed to warm to room temperature, at which point complete conversion of the amine to chloroacetamide is found by thin layer chromatography (TLC) analysis. The reaction is quenched with the addition of 10 mL brine, and the product is extracted in five portions of ethyl acetate (5×50 mL). The combined organic layers are dried over anhydrous magnesium sulfate, filtered, and concentrated in vacuo. The crude product is re-dissolved in dichloromethane and purified by silica gel flash chromatography (gradient 0% 80% EtOAc/hexanes) to afford the final chloride intermediary 4 as a soft off white solid. Depicted are characterizations of the intermediary 4.

FIG. 22 shows proteasome-activating peptides and proteasome-inhibiting peptide mimics for both proteasome inhibition and activation.

FIG. 23 shows competitive disabling through an activator inhibitor pair uses a C-terminal proteasome activator sequence such as Arg-Arg-Arg-Arg-Pro-Pro-Pro and an N-terminal proteasome inhibitor fragment, such as that which is found in the commercial boronic acid inhibitor Bortezomib (an additional inhibiting component warhead can be added to further the effect). These two can compete with each other to set a normal/healthy proteasomal activity. The way this is implemented depends on the competitive strength of the two when conjugated—where one dominates (the primary) over the other (the secondary). The balance would be shifted depending on the nature of the linker which contains the biorecognition element. In a simple implementation, the biorecognition and cleavage of the biorecognition element in the linker would sever the two and allow them to act independently, enabling the secondary to counter and incapacitate the primary's effect.

FIG. 24 shows characterization data. The adaptive activator inhibitor pair construct in this example is a peptide with the biorecognition sequence for prostasin and flanking glycine's tethering the two-altogether forming the linker GQARGG. It is synthesized according to standard HBTU/FMOC solid phase peptide synthesis (SPPS) chemistry and cleaved according to standard TFA/TIPS/thiol scavenging conditions. Depicted is characterization of the peptide sequence LFLGQARGGRRRRPPP.

FIG. 25 shows characterization data. The adaptive proteasome activator-inhibitor pair incubated with recombinant prostasin enzyme and is analyzed by LC/MS to show the independent activator byproduct for the competition. This is not observed in absence of the enzyme incubation.

FIG. 26 shows the adaptive drug peptide contains a peptide based ligand for TrkA such as the peptide sequence IENPQYFSDA, tethered to another peptide sequence that functions as the VHL/E3 ligase recruiter sequence, such as ALAPYIP, which drives ubiquitinylation and subsequent proteasomal degradation of TrkA, ultimately inhibiting phosphorylative activation of TrkA. These two different peptide biorecognition domains are tethered together with a protease cleavable biorecognition sequence such as GQARGG, which can be cleaved into GQAR-and-GG by an enzyme prostasin. Upon cleavage, the ligands are separated, and no longer are able to implement their function of targeted TrkA degradation through proximity to a VHL/E3 ligase ligand. This may be further elaborated with a cell penetrating peptide sequence to facilitate cell entry, such as RRRRRRRR, as was used in the source (non-adaptive) PROTAC publication (Hines, J., Gough, J. D., Corson, T. W., & Crews, C. M. (2013)).

FIG. 27 shows characterization data. The entire adaptive PROTAC construct in this example is a peptide, it was synthesized according to standard HBTU/FMOC solid phase peptide synthesis (SPPS) chemistry and cleaved according to standard TFA/TIPS/thiol scavenging conditions. Depicted is LC-MS (ESI-MS) of the peptide.

FIG. 28 shows characterization data. The adaptive peptide PROTAC is incubated with recombinant prostasin enzyme and is analyzed by LC/MS to show the separated TrkA ligand byproduct of the dismemberment. This is not observed in absence of the enzyme incubation.

FIG. 29 shows application of adaptive peptide PROTAC shows reduction in cell numbers for different breast cancer lines of different subtypes (MCF7, MDA-MB-231 and MDA-MB-468) without significant change in normal cell line (MCF10a).

FIG. 30 shows application of adaptive peptide PROTAC shows effect of varying applied dose (1×, half concentration and a quarter concentration as well as a no dose control C) on cancer line cell number reduction at 1 hr.

FIG. 31 shows application of adaptive peptide PROTAC shows effect of varying applied dose (1×, half concentration and a quarter concentration as well as a no dose control C) on cancer line cell number reduction at 24 hrs.

FIG. 32 shows adaptive PROTAC uses a bifunctional small molecule targeting strategy, wherein one of the small molecules may be (+)-JQ-1, as a BET bromodomain ligand, and the other small molecule may be an aminothalidomide, which is an E3 ligase ligand. These two small molecules, in a conventional PROTAC, are conjoined with a biochemically inert linker. However, in this adaptive construct, these two components incorporate the release trigger architecture and the biorecognition sequence to engage the dismemberment (seperation) of the two ligands. Specifically, they use an aminodiacid linker connected to a hydroxybenzyl carbamate peptide ester. Cleavage after recognition of the biorecognition element (depicted as peptide for example) triggers a spontaneous 1,4-methide elimination of the hydroxybenzyl carbamate moiety, liberating a primary amine, which can subsequently engage a distal ester in a 5- or 6-membered O,N-acyl shift, resulting in the release of a hydroxyethyl ester of JQ-1 and the formation of a δ- or γ-lactone. When connected to each other through a branched linker such as the example shown, the proximity effect of the E3 ligase ligand and BET bromodomain ligand to each other ultimately cause degradation of the BET bromodomain protein; however, after such a cleavage event, the separation of the two components renders the compound incapacitated as a PROTAC in its ability to degrade BET bromodomain proteins.

FIG. 33 shows an embodiment of a design scheme, at baseline the full adaptive drug has a linear, uncharged sulfate which shouldn't interfere with the drug active's availability to affect the target. However, when the recognition sequence is recognized and reacted with (cleavage), a similar release trigger architecture causes a cascade of reactions in the linker resulting in an intramolecular S_{N}2 process. The downstream effect here is to ultimately yield a sulfonated drug which is anionic and therefore easily cleared.

FIG. 34 shows an embodiment of a synthesis route for an adaptively clearable rapamycin. Intermediary steps are numbered.

FIG. 35 shows the characterization data for FIG. 34. To produce intermediary 2 is a similar reaction step 2 in FIG. 18. For other key intermediaries in the reaction, to a 20 mL oven dried round bottom flask containing a Teflon stir bar is added alcohol intermediary 2 (506 mg, 1.61 mmol, 1 equiv) in dry acetonitrile. 6.4 μL N-methylimidazole was added upon dissolving the starting material. Then, 314 mg CDI (2.0 mmol, 1.2 equiv) is added as a single portion. After 1 hour, an additional 19.4 mg CDI is added to fully convert alcohol intermediary 2 to an imidazole carbamate. An additional 0.4 equiv CDI and an additional 10 μL of N-methylimidazole are added to drive the reaction to completion. Upon complete conversion of intermediary 2,2-(2-aminoethoxy) ethanol (650 mg, 3.8 equiv) is added as a single portion under vigorous stirring. Within five minutes, complete conversion to ethoxyethanol intermediary 3 is observed by TLC (thin layer chromatography) analysis. The reaction was quenched with the addition of 6 mL 1 M HCl, and acetonitrile was removed in vacuo. The resulting aqueous solution is transferred to a 50 mL separatory funnel, and the product is extracted in two portions of 50 mL ethyl acetate. The resulting organic layers are dried over anhydrous magnesium sulfate, filtered, and concentrated in vacuo. The resulting compound is purified by silica gel flash chromatography to afford alcohol intermediary 3 as a translucent solid. Depicted are the characterizations of intermediary 3 as well as intermediary 3′ in the rapamycin conjugation.

FIG. 36 shows an embodiment of an alternative synthesis route for an adaptively clearable rapamycin which doesn't use the piperazine conjugated rapamycin. Intermediary steps are numbered.

FIG. 37 shows specific design of adaptive sulfonation strategy for the BET bromodomain inhibitor JQ-1. An alternative architecture with a similar mechanism is shown with adaptive phosphorylation of the JQ-1.

FIG. 38 shows amonafide drug active has some cationic nature and this is enhanced by the polycationic cell entry peptide. This enhances its mobility to both pass through the cell membrane and better intercalate into DNA. In this implementation of the reducing mobility mode the tethering between the two contains the biorecognition element (in this case for prostasin) as well as an additional linker component. When recognized and acted upon, the cleavage at the biorecognition element site occurs in a way such that the amonafide retains the biorecognition element and an additional linker piece but loses the cell entry peptide. Without the cell entry peptide, the mobility through the cell membrane is reduced. In addition, the linker piece remaining tethered to the drug active is chosen to offset the charge of the amonafide leaving the piece charge-neutral or charge-negative. This then further lacks the electrostatic feature required for navigating to and intercalating into the DNA. Both of these forms of mobility reduction incapacitate the drug.

FIG. 39 shows synthesis routed depicted for adaptive amonafide intercalating agent. Intermediaries are numbered below each compound.

FIG. 40 shows production of intermediary 2. To a 10 mL round bottom flask with an oven dried Teflon stir bar is added amonafide HCl (120 mg, 0.4 mmol, 1 equiv) in DMF (2.0 mL) and triethylamine (0.10 mL, 1 mmol, 2.5 equiv) under nitrogen. Ethyl chlorosuccinate (85 mg). The reaction mixture is stirred for 24 hours at room temperature, at which TLC analysis indicates full conversion. Most of the DMF is subsequently removed in vacuo, and the resulting residue was loaded onto a column and purified by silica gel flash chromatography (gradient 50% 100% EtOAc in hexanes) to afford amonafide ethylsuccinamide intermediary 2 as a waxy yellow powder. Depicted are characterizations of intermediary 2.

FIG. 41 shows characterization data. Production of intermediary 3 in telescoped reaction with previous step. DMAP and triethylamine are added at room temperature. Following this DMAP (122 mg, 1.0 mmol, 3 equiv) and triethylamine (500 mg, 5.0 mmol, 15 equiv) is added at room temperature. Next, succinic anhydride (350 mg, 3.4 mmol, 10 equiv) is added as a single portion, and the reaction is stirred at room temperature for 48-52 hours. Reaction progress is followed by thin layer chromatography (TLC). Upon completion, the solvent is evaporated in vacuo and the crude reaction mixture is directly purified by silica gel flash chromatography (gradient of 100% EtOAc 4:1 EtOH:EtOAc) to afford succinate amide intermediary 3 as a yellow powder. Depicted are characterizations of intermediary 3.

FIG. 42 shows characterization data. Completion of the final step in synthesis first requires peptide GQARGGRRRRRRRG to be separately synthesized with standard HBTU/DIPEA coupling conditions on solid phase peptide synthesis. Upon completion of the synthesis, the N-terminal FMOC is deprotected by stirring in 20% v/v 4-methylpiperazine in DMF for 1 hour. The resulting Wang resin containing the N-terminal deprotected peptide is drained of excess solution and dried for 1 hour under vacuum. Subsequently, a portion of the resin calculated to contain 0.125 mmol peptide is re-swelled in DMF (HPLC grade), and HBTU (180 mg, 0.50 mmol, 4 equiv), DMAP (60 mg, 0.50 mmol, 4 equiv), and intermediary 3 (120 mg, 1.0 mmol, 4 equiv) was added. The reaction was permitted to stir in the dark at room temperature under nitrogen for 48 hours. After this, the resin is re-drained and washed with two portions of 10 mL DMF, followed by 2 portions of 10 mL methanol, followed by 1 portion 10 mL dichloromethane. Subsequently, a cleavage solution is separately prepared according to literature protocols (94% TFA v/v, 1% TIPS, 2.5% DTT w/v, 2.4% water v/v) and 10.0 mL of this is added to the peptide conjugated resin. The resulting yellow solution is permitted to stir for 2 hours, at which point the resin is drained and washed with an additional 10 mL TFA and 25 mL methanol. The resulting solution was concentrated in vacuo. After removal of a majority of the water and TFA, the reaction mixture is extracted in 10 mL methanol from 10 mL 1:1 hexanes:diethyl ether, and the methanolic layer is once again concentrated in vacuo. The resulting product is purified by preparative HPLC. Depicted are characterizations of the final compound.

FIG. 43 shows application of adaptive intercalating agent shows effect of applied dose varying (1×, a third, a ninth and a twenty-seventh concentration as well as a no dose control C) isolated cDNA during qPCR amplification. Only control shows amplification, while all other doses show negligible amplification (cDNA replication).

FIG. 44 shows application of adaptive intercalating agent shows reduction in cell numbers for different breast cancer lines of different subtypes (MCF7, MDA-MB-231 and MDA-MB-468) without significant change in normal cell line (MCF10a).

FIG. 45 shows application of adaptive intercalating agent shows effect of varying applied dose (1× and a third concentration as well as a no dose control C) on cancer line cell number reduction at 1 hr.

FIG. 46 shows application of adaptive intercalating agent shows effect of varying applied dose (1× and a third concentration as well as a no dose control C) on cancer line cell number reduction at 24 hrs.

FIG. 47 shows the adaptive drug designed to naturally release the commercial drug Bobcat 339 by auto-hydrolysis. Bobcat 339 functions normally using ambient copper as a cofactor. Recognition and reaction of the biorecognition element uses the release trigger mechanism to then also release a hydroxybipyridyl ligand. This will outcompete Bobcat 339 for the needed copper cofactor, thus incapacitating the drug.

FIG. 48 shows an example of a primary hydroxyl-containing small drug active auto hydrolyses to have a biological effect, though slowly. However, upon biorecognition sequence (peptide) recognition and reaction, the fast release trigger mechanism triggers a 6-exo-tet reaction to also dismember and rearrange the hydroxyl group from the rest of the drug component, thereby incapacitating it. An example of such a drug is the discussed inhibitor JQ-1.

FIG. 49 shows mobility reduction. After recognition and cleavage of the biorecognition element (peptide), the release trigger elimination leads to formation of a benzylic diazo, which upon extrusion of nitrogen provides a carbene. This then can react via C—H insertion into its surrounding molecules in the environment via a Shapiro/Bamford Stevens like Tosylhydrazone elimination (note the addition of adjacent germinal methyls to prevent elimination as in a Bamford). This links the drug component to those surrounding molecules thus preventing it from moving towards the biological target and thus incapacitating the drug.

FIG. 50 shows schematics of the adaptive drug examples that depict roles and actions of the biorecognition element, drug active element, and different modality of incapacitation mechanism. Engagement of incapacitation mechanism initiated by recognition of the biorecognition element is also depicted.

### DETAILED DESCRIPTION

In systems engineering, a central tenet of designing a stable control system is to use negative feedback. This means tuning down an intervening stimulus as the system approaches closer to the desired state. This prevents unwanted consequences from overshooting or undershooting the desired state. The automation of this process is achieved by a controller or regulator which measures an indicative feature of the desired state and then inversely tunes the stimulus. This is also referred to as an adaptive system and is often more precise, faster acting and more scalable than manual manipulation of the system.

Negative feedback is critical for drugs or other medical interventions because of their potential for side and off target effects from underdosing or overdosing. It is, however, by and large done by manual manipulation—a patient report systems, labs run diagnostics on the patient, a physician analyzes the results and they modify prescription for a drug, the patient takes the drug and waits for improvement or symptoms, then this cycle repeats as treatment and the disease progresses. However, this manual manipulation is costly, requires a lot of medical infrastructure, can be too slow and out of sync with the patient's needs and ultimately involves a lot of trial and error which can mean major side and off target effects for the patient.

Contrast this with the natural regulatory processes (immune, metabolic, etc) of the body which are indeed adaptive in their actions. They adaptively intervene and regulate a tissue, organ or the whole body based on its current state, and thus enable a stable transition to a healthy state and function-processes known as homeostatic regulation. While this natural regulation works for many stresses to the systems, diseases can stress the system in a way or to an extent that exceeds the body's homeostatic recovery capacity. It is then that the body requires external support through medicine and therapy. Of course, then for an equally stable recovery from these advanced diseased states—without overshooting/undershooting, side and off target effects—an optimal medicinal intervention would also be adaptive, like the body's homeostatic intervention. Presented here, is a novel paradigm of adaptive drug logics that implement the principle of negative feedback to autotune medicines to make them more precise, real time, and homeostatic in their effect on the body.

Negative feedback or adaptive systems have three fundamental components—a sensing unit, a stimulus unit and a regulator that inversely ties the two. The sensing unit measures the level of a desired outcome and the stimulus is meant to drive the system towards the desired outcome. Thus the natural relationship between the two should be inverted (as more the desired outcome is sensed the stimulus should be lessened and vice versa). This same conjugation of multiple components will also be used for the adaptive drugs that implement negative feedback.

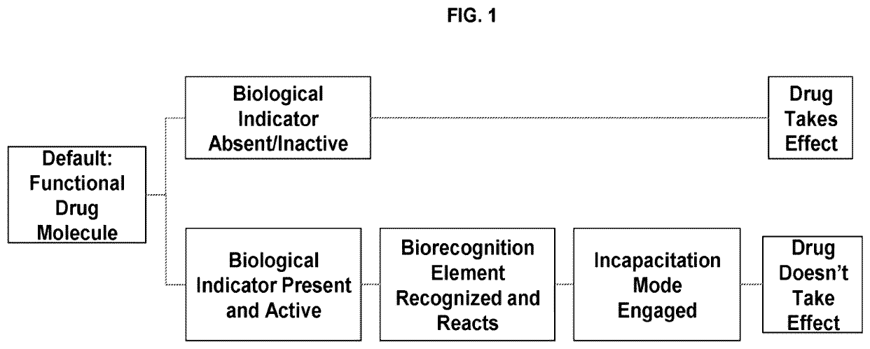

Conjugate drugs currently exist primarily for targeting a drug active to a disease marker. Their design involves the use of a sensing component for the diseased marker, the drug active and a chemical linker just physically tying the two. We build off this compartmentalized structure but replace the disease marker sensing component with a healthy state sensing component (biorecognition element), keep the drug active and replace the linker with a regulator (incapacitation mechanism) that ties the two physically but also ties the drug's functional capacity to the recognition or lack-there-of of the biorecognition component. This tying, as mentioned, will implement an inverse mechanics—when the healthy state defined by a biological indicator recognizes and reacts with the biorecognition element, the drug active will lose its capacity to function through the incapacitation mechanism. FIG. 1 shows a simple schematic of the adaptive drug's behavior.

The biorecognition element of the present invention comprises a substrate that is recognizable or identifiable by the specific biological indicator. Enzymes make a good class of biological indicators because they can be specific and many of their target substrates are known. The enzyme can be located anywhere within the subject, for instance extracellular, cell membrane embedded, cytoplasmic, nuclear membrane embedded, or nuclear. In many cases the enzyme will cleave or alter a specific bond in the substrate—this can then be used to engage the incapacitation mechanism. For example, the enzyme can be a hydrolase, an oxidoreductase, a transferase, a translocase, a lyase, a ligase, or an isomerase. In these cases, the substrate can be designed to comprise a sequence and/or a specific bond recognized by an enzyme, and the enzyme will break the bond to trigger the incapacitation component. These enzymes of course can be broken down into further categories of specific substrate types. For example substrates for hydrolases include, but are not limited to, peptide sequences for proteases, oligonucleotide sequences for nucleases, lipid or lipidic ester sequences for lipases, phosphate groups for phosphatases. Other hydrolases and substrates with enzyme-substrate specificity are also suitable for the present invention. Proteases include enzymes such as prostasin, matriptase, and CYLD lysine 63 deubiquitinase. Esterase include enzymes such as phosphodiesterase 2A. Phosphatases include enzymes such as phosphatidylinositol-3,4,5-trisphosphate 3 phosphatase, phosphatidic acid phosphatase type 2B, or inositol polyphosphate-5 phosphatase.

Other classes of enzymes which can be used are oxidoreductases or a transferases that transfers bonds or electrons from one substrate to another. In such case, the enzyme would recognize the biorecognition element and transfer a specific bond or electrons to engage the incapacitation mechanism. Example transferases include methyltransferase and 3-hydroxy-3-methylglutaryl-CoA synthase 2. Example oxidoreductases include aldehyde dehydrogenase 2 family (mitochondrial).

The incapacitation mechanism can be implemented through multiple modes by which the drug is incapacitated. Some example modes are the following 1) Decoy Presentation 2) Competitive Disabling 3) Drug Dismemberment 4) Increased Clearability and 5) Mobility Reduction. Example adaptive drugs for each mode are given in the following sections

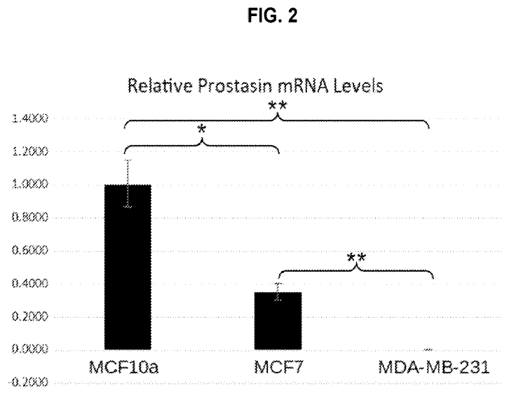

For these examples a test case is shown where the biological indicator for a healthy state is the enzyme prostasin. It serves multiple functions that enforce healthy epithelial functions, such as nutrient absorption through sodium ion channel (ENaC) maintenance and barrier enforcement through tight junction facilitation [Szabo, R., & Bugge, T. H. (2011)]. The epithelial identity is compromised with diseases, for instance in carcinomas (epithelium derived cancers) as the cells lose their epithelial nature and transition to more mesenchymal nature (EMT) [Martin, C. E., & List, K. (2019)]. Thus prostasin can act as a marker of healthy, non-cancerous epithelial tissue. This is known from the literature but further validated in FIG. 2. In the examples given, the biorecognition element for prostasin is a known peptide substrate recognized by prostasin (as the enzyme is a protease). Specifically the sequence QAR with an addition linking glycine (G) is used [Duru, N., Pawar, N. R., Martin, E. W., Buzza, M. S., Conway, G. D., Lapidus, R. G., . . . & Antalis, T. M. (2022)], but other known biorecognition sequences are also known including KHYR or PRLR [Shipway, A., Danahay, H., Williams, J. A., Tully, D. C., Backes, B. J., & Harris, J. L. (2004)]. In further validation of this, a 7-Amino-4-methylcoumarin (AMC) reporter was attached to the QAR sequence. Upon cleavage by prostasin from cells, AMC molecule fluoresces in the with ex: 341 nm em: 441 nm. Normal line MCF10a exhibits substantially blue signal compared to MCF7 and even more so compared to MDA-MB-231 cancer cell lines, correlating to recognition and cleavage of the test substrate. Images not shown here.

As peptide synthesis can be done routinely, generically and at scale with industry grade peptide synthesizers, it is trivial to synthesize and replace the QARG biorecognition element with any other sequence for prostasin or another enzyme.

The inventors have developed a mechanism of adaptive medicine that utilizes a chemical logic to titrate drug efficacy in situ based on the real-time conditions of the cells, tissue, or system. In one aspect, the inventors have developed a regulator construct comprising a drug component that inversely responds to the level of a selected indicator enzymatic activity. The regulator construct works to inactivate or incapacitate an active drug in the presence of the enzyme. Moreover, the more favored enzymatic presence and activity, the more of the drug will be deactivated by the regulator construct. Conversely, the more disease progression limiting the enzymatic presence or impairing its function, the more effective the drug will be. The drug target and the indicator enzyme do not need to be related, so instantiations can be designed to choose the best indicator of the disease and can affect a completely different process as a result. As enzymes are the fundamental catalysts of biochemical reactions, there are a multitude of disease applications where specific enzymes are not produced and thus this occurrence can be used as an indicator to guide the construct logic. The inversely responding logic has powerful and novel utility. For one, it opens up the possibility of now utilizing the vast space of markers inversely (negatively) correlated with disease and thus positively correlated with health, which, by and large, are not currently focused on; most contemporary solutions with some selectively only search for, and are designed around, markers positively correlated disease. Also using impaired function (inversely correlated markers as our aforementioned limited presence of a particular enzyme) as an indicator is, in many ways, more natural to disease treatment than searching for added, unwanted functions (directly correlated markers). That is the nature of disease itself, fundamentally to impair function.

Drugs by their nature are designed and optimized to increase efficacy, so instead of trying to narrow, contain or isolate its effects to prevent side- or off-target effects while retaining some efficacy, as current strategies do, it can be more natural and effective to have an optimally efficacious drug component and a mechanism to inactivate or incapacitate it when not needed. Furthermore, this logic establishes a negative feedback loop between the drug and the biology, which is well known in control theory to more stably drive and reinforce an optimal state or function, in this consideration healthy biological cells, tissues or other body systems. Ultimately, this negative feedback construct allows for the establishment of a set dose to be administered with an adaptive efficacy or efficacious dose in situ that is naturally auto-tuned to the current state of the disease for that patient at that time. In this sense, this solution is the biological analogy to real-time feedback and modulation in other fields-like servos in mechanical systems or closed loop gains from op-amps in electronics. It also allows a more appropriate and comprehensive gradation of drug administration rather than the discrete stage defined or one-size-fits-all dosing currently in practice.

To implement the adaptive medicine as described above, the present invention provides a regulator construct which is a chemical structure comprising: (a) a biorecognition component comprising a substrate recognized by an enzyme, (b) incapacitation component, covalently linked to the biorecognition component, and (c) a drug component covalently linked to the incapacitation component, wherein the incapacitation component provides a mechanism to inactivate or incapacitate the drug component upon biorecognition and reaction of the biorecognition component with the indicator enzyme, and wherein the drug is active in the chemical structure, but inactive or incapacitated after recognition.

The chemical structure of the present invention can be applied to cells, tissue or body systems of a subject, in which there exists an indicator enzyme which 1) decreases in quantity or activity as a disease progresses, and 2) has a specific substrate recognized by that enzyme.
biorecognition element is a specific substrate that, upon recognition and reaction by a target enzyme, triggers an incapacitation mechanism to neutralize (inactivate or incapacitate) the drug component.

### Auto-Regulating Compound

The compounds of the present invention can be an auto-regulating compound. Auto-regulating compounds are compounds which are functional or incapacitated dependent upon response to a biological stimulus. In a non-limiting example, if there is a high level of a biological stimulus such as higher concentrations of an enzyme which indicates disease is being treated or mitigated, then the auto-regulating compound's incapacitation mechanism is activated, which incapacitates the active drug. If there is a low level or no biological stimulus, such as an absence of the enzyme, then it indicates there is disease progression, and the auto-regulating compound stays active to treat the disease or disorder.

In some embodiments, is a chemical structure comprising: (a) a biorecognition component comprising a substrate recognized by an enzyme, (b) an incapacitation component, covalently linked to the biorecognition component, and (c) a drug component covalently linked to the incapacitation component; wherein the incapacitation component provides an incapacitation mechanism to inactivate or incapacitate the drug component upon substrate sequence recognition and reaction with the enzyme, and the drug is active in the chemical structure, but inactive or incapacitated after the recognition and reaction.

In some embodiments, is a chemical structure comprising: (a) a biorecognition component comprising a substrate recognized by an enzyme, (b) an incapacitation component, covalently linked to the biorecognition component, and; (c) a drug component covalently linked to the incapacitation component; wherein the incapacitation component provides an incapacitation mechanism to incapacitate the drug component upon substrate sequence recognition and reaction with the enzyme, and the drug is capable to execute its biological effect, but incapacitated after the recognition and reaction.

In some embodiments is an auto-regulating compound comprising a biorecognition component comprising: (a) a substrate that is recognized by an enzyme; (b) an incapacitation component; and (c) a drug component comprising a therapeutic agent, wherein the drug component is active in the auto-regulating compound; wherein: the incapacitation component is covalently linked to the biorecognition component and the drug component; and wherein when the enzyme reacts with the biorecognition component, a covalent bond tethering the biorecognition component is cleaved, triggering an incapacitation mechanism to incapacitate the drug component.

In some embodiments is a auto-regulating compound comprising: (a) a biorecognition component comprising a substrate that is recognized by an enzyme; (b) an incapacitation component; and (c) a drug component comprising a therapeutic agent, wherein the drug component is active in the auto-regulating compound; wherein: the incapacitation component is covalently linked to the biorecognition component and the drug component such that the biorecognition component is on one end of the incapacitation component and the drug component is on another end of the incapacitation component; and wherein when the enzyme reacts with the substrate, the biorecognition component is released from the incapacitation component, which triggers an incapacitation mechanism to incapacitate the drug component.

In some embodiments, auto-regulating compound comprising: (a) a biorecognition component comprising a substrate that is recognized by an enzyme; (b) an incapacitation component; and (c) a drug component comprising a therapeutic agent, wherein the drug component retains its biological functional capacity in the auto-regulating compound; wherein: the incapacitation component is covalently linked to the biorecognition component and the drug component; and wherein when the biorecognition component binds to the enzyme, the biorecognition component is released from the incapacitation component, and triggers an incapacitation mechanism, wherein the incapacitation mechanism is the diversion, out-competition, breakdown, clearance, or mobility impairment of the auto-regulating compound, resulting in the incapacitation of the drug component.

In some embodiments is a auto-regulating compound comprising: (a) a recognition component comprising a substrate that is recognized by an enzyme; (b) an incapacitation component; and (c) a drug component comprising a therapeutic agent, wherein the drug component is active in the auto-regulating compound; wherein: the incapacitation component is covalently linked to the biorecognition component and the drug component such that the biorecognition component is on one end of the incapacitation component and the drug component is on another end of the incapacitation component; and wherein when the biorecognition component binds to the enzyme, the biorecognition component is released from the incapacitation component, and triggers an incapacitation mechanism wherein the drug component becomes inactive.

In some embodiments, the chemical structure or the auto-regulating compound has a structure of A-B-C, wherein A is the biorecognition component, B is the incapacitation component, and C is the drug component.

The enzymes useful in the present invention can be any suitable enzyme known by one of skill in the art. The enzymes are an indicator of the disease or disorder. The enzyme can be an indicator for a specific application. In some embodiments, the enzymes are absent or in a low concentration when the disease is progressing. In some embodiments, the enzymes are in a higher concentration when the disease is being treated or mitigated, and/or healthy cells or tissue is present. In some embodiments, the enzymes are in a higher concentration when the disease is being treated or mitigated.

In some embodiments, the chemical structure or the auto-regulating compound has a structure of A-B-C, C-A-B, C-A-B-C′, wherein A is the biorecognition component, B is the incapacitation component, C is the drug component, and C′ is a further additional drug component. In some embodiments, the chemical structure or auto-regulating compound has a structure of A-B-C, wherein A is the biorecognition component, B is the incapacitation component, and C is the drug component.

In some embodiments, the enzyme cleaves or alters a covalent bond between the biorecognition component and the incapacitation component. In some embodiments, the enzyme cleaves or alters a covalent bond tethering the biorecognition component to engage the incapacitation mechanism. In some embodiments, the enzyme cleaves the covalent bond. In some embodiments, the enzyme alters the covalent bond between the biorecognition component and the incapacitation component.

In some embodiments, the enzyme cleaves the covalent bond. In some embodiments, the covalent bond is an amide bond, an ester bond, or a disulfide bond. In some embodiments, the covalent bond is an amide bond. In some embodiments, the covalent bond is an ester bond. In some embodiments, the covalent bond is a disulfide bond.

In some embodiments, the enzyme alters the covalent bond between the biorecognition component and the incapacitation component by transfer of electrons or bonds from one substrate to another.

In some embodiments, the enzyme is a hydrolase, an oxidoreductase, a transferase, a translocase, a lyase, a ligase, or an isomerase. In some embodiments, the enzyme is hydrolase. In some embodiments, the enzyme is a translocase. In some embodiments, the enzyme is a lyase. In some embodiments, the enzyme is a ligase. In some embodiments, the enzyme is an isomerase.

In some embodiments, the hydrolase is a protease, a nuclease, a lipase, a phosphatase, or an esterase. In some embodiments, the hydrolase is a protease. In some embodiments, the hydrolase is a nuclease. In some embodiments, the hydrolase is a lipase. In some embodiments, the hydrolase is a phosphatase. In some embodiments, the hydrolase is an esterase.

In some embodiments, the protease is a serine protease, a cysteine protease, a threonine protease, an aspartic protease, a glutamic protease, a metalloprotease, or an asparagine peptide lyase. In some embodiments the protease is a serine protease. In some embodiments, the protease is a cysteine protease. In some embodiments, the protease is a threonine protease. In some embodiments, the protease is an aspartic protease. In some embodiments, the protease is a glutamic protease. In some embodiments, the protease is a metalloprotease. In some embodiments, the protease is an asparagine peptide lyase. In some embodiments, the protease is prostasin, matriptase, or CYLD lysin 63 deubiquitinase.

In some embodiments, the nuclease is an endonuclease, an exonuclease, a DNase, a RNase, a topoisomerase, a recombinase, a ribozyme, or an RNA splicing enzyme. In some embodiments, the nuclease is endonuclease. In some embodiments, the nuclease is an exonuclease. In some embodiments, the nuclease is a DNase. In some embodiments, the nuclease is RNase. In some embodiments, the nuclease is topoisomerase. In some embodiments, the nuclease is a ribozyme. In some embodiments, the nuclease is an RNA splicing enzyme. In some embodiments, the nuclease is BAL 31 nuclease, Yatalase, cryonase cold-active nuclease, exonuclease I, exonuclease III, nicrococcal nuclease, mung bean nuclease, recombinant DNase (I), ribonuclease H, or S1 nuclease.

In some embodiments, the lipase is bile salt-dependent lipase, pancreatic lipase, lysosomal lipase, hepatic lipase, lipoprotein lipase, hormone-sensitive lipase, gastric lipase, endothelial lipase, or lingual lipase. In some embodiments, the phosphatase is phosphatidylinositol-3,4,5-trisphosphate 3-phosphatase, phosphatidic acid phosphatase type 2B, or inositol polyphosphate-5-phosphatase. In some embodiments, the esterase is acetylesterase or phosphodiesterase 2.

In some embodiments, the enzyme is oxidoreductase or transferase. In some embodiments, the enzyme is oxidoreductase. In some embodiments, the enzyme is transferase.

In some embodiments, the oxidoreductase is aldehyde dehydrogenase-2. In some embodiments, the transferase is methyltransferase or 3-hydroxy-3-methylglutaryl-CoA synthase 2.

In some embodiments, the enzyme is located in a subject's extracellular space, cell membrane, cytoplasm, nucleus, or nuclear membrane. In some embodiments, the enzyme recognizes the substrate of the biorecognition component. In some embodiments, the substrate comprises a peptide sequence or a biorecognition element, wherein the enzyme recognizes the peptide sequence or the biorecognition element. In some embodiments, the peptide sequence is recognized by protease. In some embodiments, the peptide sequence is QAR.

In some embodiments, the biorecognition element is an oligonucleotide, lipid or lipidic ester, or phosphate groups. In some embodiments, the biorecognition element is an oligonucleotide. In some embodiments, the biorecognition element is a lipid or lipidic ester. In some embodiments, the biorecognition element is a phosphate group.

In some embodiments, the enzyme cleaves or alters a covalent bond between the biorecognition component and the incapacitation component to trigger the incapacitation mechanism.

In some embodiments, the auto-regulating compound is a structure of Formula (II):

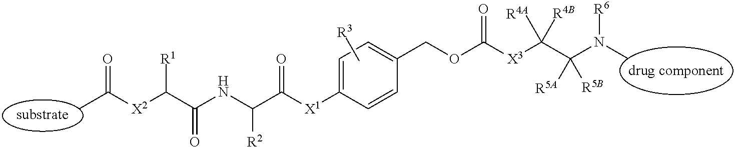

wherein:
   X¹, X², and X³ are each independently O, NH, or S;
   R¹, R², R³, R^{4A}, R^{4B}, R^{5A}, R^{5B}, and R⁶ are each independently H or C₁₋₆ alkyl; substrate is a peptide; and
   drug component is a therapeutic agent.

X¹ can be any suitable electronegative atom. In some embodiments, X¹ is O, NH, or S. In some embodiments, X¹ is O or NH. In some embodiments, X¹ is O or S. In some embodiments, X¹ is NH or S. In some embodiments, X¹ is O. In some embodiments, X¹ is NH. In some embodiments, X¹ is S.

X² can be any suitable electronegative atom. In some embodiments, X² is O, NH, or S. In some embodiments, X² is O or NH. In some embodiments, X² is O or S. In some embodiments, X² is NH or S. In some embodiments, X² is O. In some embodiments, X² is NH. In some embodiments, X² is S.

X³ can be any suitable electronegative atom. In some embodiments, X³ is O, NH, or S. In some embodiments, X³ is O or NH. In some embodiments, X³ is O or S. In some embodiments, X³ is NH or S. In some embodiments, X³ is O. In some embodiments, X³ is NH. In some embodiments, X³ is S.

R¹ can be any suitable functional group. In some embodiments, R¹ is H or C₁₋₆ alkyl. In some embodiments, R¹ is H or C₁₋₃ alkyl. In some embodiments, R¹ is H or methyl. In some embodiments, R¹ is H. In some embodiments, R¹ is C₁₋₃ alkyl. In some embodiments, R¹ is methyl or ethyl. In some embodiments, R¹ is methyl. In some embodiments, R¹ is ethyl.

R² can be any suitable functional group. In some embodiments, R² is H or C₁₋₆ alkyl. In some embodiments, R² is H or C₁₋₃ alkyl. In some embodiments, R² is H or methyl. In some embodiments, R² is H. In some embodiments, R² is C₁₋₃ alkyl. In some embodiments, R² is methyl or ethyl. In some embodiments, R² is methyl. In some embodiments, R² is ethyl.

R³ can be any suitable functional group. In some embodiments, R³ is H or C₁₋₆ alkyl. In some embodiments, R³ is H or C₁₋₃ alkyl. In some embodiments, R³ is H or methyl. In some embodiments, R³ is H. In some embodiments, R³ is C₁₋₃ alkyl. In some embodiments, R³ is methyl or ethyl. In some embodiments, R³ is methyl. In some embodiments, R³ is ethyl.

R^{4A} can be any suitable functional group. In some embodiments, R^{4A} is H or C₁₋₆ alkyl. In some embodiments, R^{4A} is H or C₁₋₃ alkyl. In some embodiments, R^{4A} is H or methyl. In some embodiments, R^{4A} is H. In some embodiments, R^{4A} is C₁₋₃ alkyl. In some embodiments, R^{4A} is methyl or ethyl. In some embodiments, R^{4A} is methyl. In some embodiments, R^{4A} is ethyl.

R^{4B} can be any suitable functional group. In some embodiments, R^{4B} is H or C₁₋₆ alkyl. In some embodiments, R^{4B} is H or C₁₋₃ alkyl. In some embodiments, R^{4B} is H or methyl. In some embodiments, R^{4B} is H. In some embodiments, R^{4B} is C₁₋₃ alkyl. In some embodiments, R^{4B} is methyl or ethyl. In some embodiments, R^{4B} is methyl. In some embodiments, R^{4B} is ethyl.

R^{5A} can be any suitable functional group. In some embodiments, R^{5A} is H or C₁₋₆ alkyl. In some embodiments, R^{5A} is H or C₁₋₃ alkyl. In some embodiments, R^{5A} is H or methyl. In some embodiments, R^{5A} is H. In some embodiments, R^{5A} is C₁₋₃ alkyl. In some embodiments, R^{5A} is methyl or ethyl. In some embodiments, R^{5A} is methyl. In some embodiments, R^{5A} is ethyl.

R^{5B} can be any suitable functional group. In some embodiments, R^{5B} is H or C₁₋₆ alkyl. In some embodiments, R^{5B} is H or C₁₋₃ alkyl. In some embodiments, R^{5B} is H or methyl. In some embodiments, R^{5B} is H. In some embodiments, R^{5B} is C₁₋₃ alkyl. In some embodiments, R^{5B} is methyl or ethyl. In some embodiments, R^{5B} is methyl. In some embodiments, R^{5B} is ethyl.

R⁶ can be any suitable functional group. In some embodiments, R⁶ is H or C₁₋₆ alkyl. In some embodiments, R⁶ is H or C₁₋₃ alkyl. In some embodiments, R⁶ is H or methyl. In some embodiments, R⁶ is H. In some embodiments, R⁶ is C₁₋₃ alkyl. In some embodiments, R⁶ is methyl or ethyl. In some embodiments, R⁶ is methyl. In some embodiments, R⁶ is ethyl.

In some embodiments, R¹, R², R³, R^{4A}, R^{4B}, R^{5A}, R^{5B}, and R⁶ are each independently H or C₁₋₃ alkyl. In some embodiments, R⁶ is H or methyl.

The substrate can be any suitable substrate recognized by an enzyme. In some embodiments, the substrate is a peptide or a biorecognition element. In some embodiments, the substrate is a peptide. In some embodiments, the peptide has the following sequence: QARG. In some embodiments, the substrate is the biorecognition element. In some embodiments the biorecognition element is an oligonucleotide, lipid or lipidic ester, or phosphate groups.

The drug component can be any suitable drug component described herein. In some embodiments, the drug component is a therapeutic agent. In some embodiments, the drug component comprises the subcomponents described herein. In some embodiments, the drug component is:

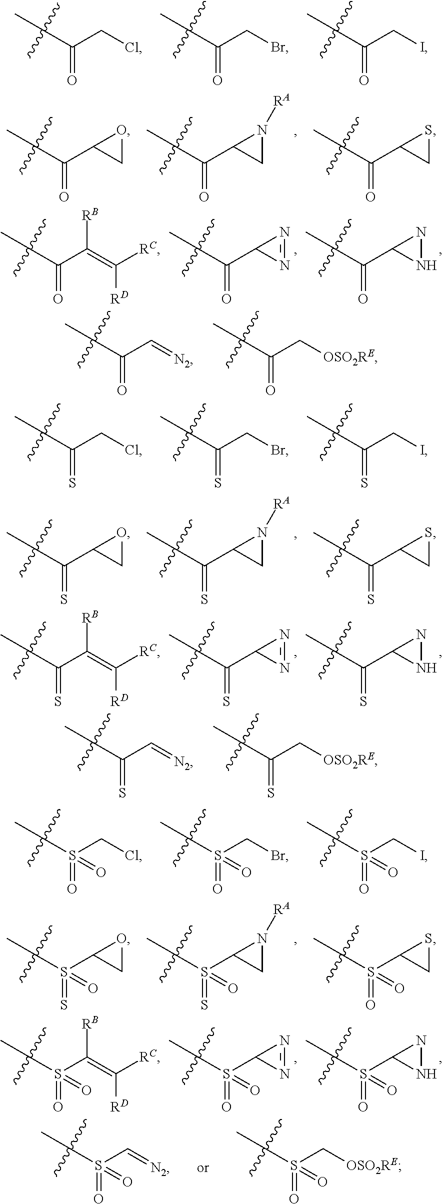

wherein: R^{A}, R^{B}, R^{C}, R^{D} and R^{E} are each independently selected from H and C₁₋₆ alkyl.

### Recognition Component

A biorecognition component of the present invention comprises a substrate that is recognizable or identifiable by the specific indicator enzyme for the chosen indication or application. The enzyme can be located anywhere within the subject, for instance extracellular, cell membrane embedded, cytoplasmic, nuclear membrane embedded, or nuclear. In a preferred embodiment, the enzyme cleaves or alters a specific bond in the substrate. For example, the enzyme can be a hydrolase, an oxidoreductase, a transferase, a translocase, a lyase, a ligase, or an isomerase. In these cases, the substrate can be designed to comprise a sequence and/or a specific bond recognized by an enzyme, and the enzyme will break the bond to trigger the incapacitation component.

The biorecognition component can comprise an antibody, nucleic acid, or substrate that is recognized by an enzyme. In some embodiments, the biorecognition component can comprise an antibody that is recognized by an antigen. In some embodiments, the biorecognition component can comprise a nucleic acid that is recognized by another nucleic acid or protein.

The biorecognition component comprises a substrate that is recognizable or identifiable by the enzymes described herein. In some embodiments, the substrate can be a peptide, protein, or biorecognition element. In some embodiments, the substrate is a peptide. In some embodiments, the substrate is a peptide that binds to an enzyme described herein. In some embodiments, the substrate is a protein that binds to an enzyme described herein. In some embodiments, the substrate is recognized by a hydrolase. In some embodiments, the substrate is recognized by protease or phosphatase. In some embodiments, the substrate is a biorecognition element. In some embodiments, the biorecognition element is an oligonucleotide, lipid or lipidic ester, or phosphate group.

In some embodiments, the biorecognition element is an oligonucleotide. In some embodiments, the oligonucleotide is an antisense oligonucleotide or aptamer. In some embodiments, the biorecognition element is a lipid or lipidic ester. In some embodiments, the biorecognition element is a phosphate group.

In one preferred embodiment, the enzyme is a hydrolase. Substrates and their corresponding hydrolases suitable for the present invention include, but are not limited to, peptide sequences for proteases, oligonucleotide sequences for nucleases, lipid or lipidic ester sequences for lipases, phosphate groups for phosphatases. Other hydrolases and substrates with enzyme-substrate specificity are also suitable for the present invention. For example, hydrolases include proteases such as prostasin, matriptase, and CYLD lysine 63 deubiquitinase. For example, hydrolases include esterase such as phosphodiesterase 2A. For example, hydrolases include phosphatases such as phosphatidylinositol-3,4,5-trisphosphate 3-phosphatase, phosphatidic acid phosphatase type 2B, or inositol polyphosphate-5-phosphatase.

In one embodiment, the enzyme is an oxidoreductase or a transferase that transfers bonds or electrons from one substrate to another. In such case, the enzyme recognizes the biorecognition element and triggers the incapacitation component through the transfer of the specific bond or electrons to inactivate the drug. For example, transferases include methyltransferase and 3-hydroxy-3-methylglutaryl-CoA synthase 2. For example, oxidoreductases include aldehyde dehydrogenase 2 family (mitochondrial).

One specific example is a substrate comprising a peptide sequence QARG for the enzyme prostasin or matriptase.

### Incapacitation Component

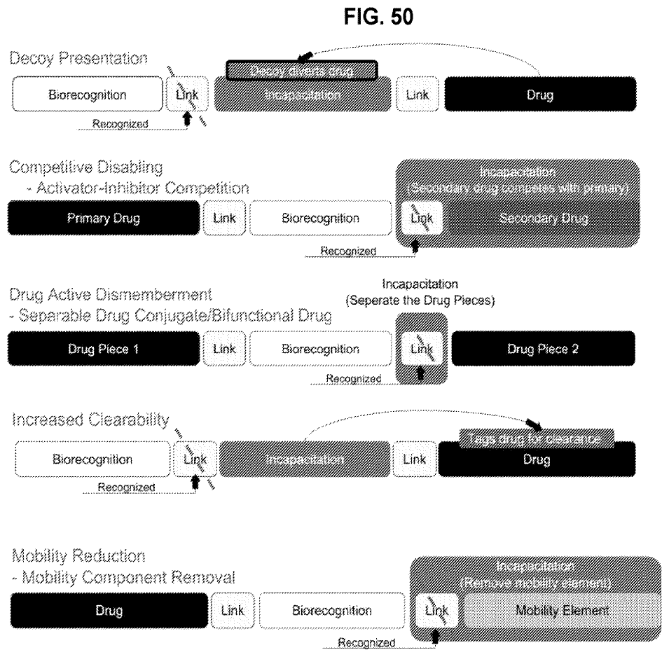

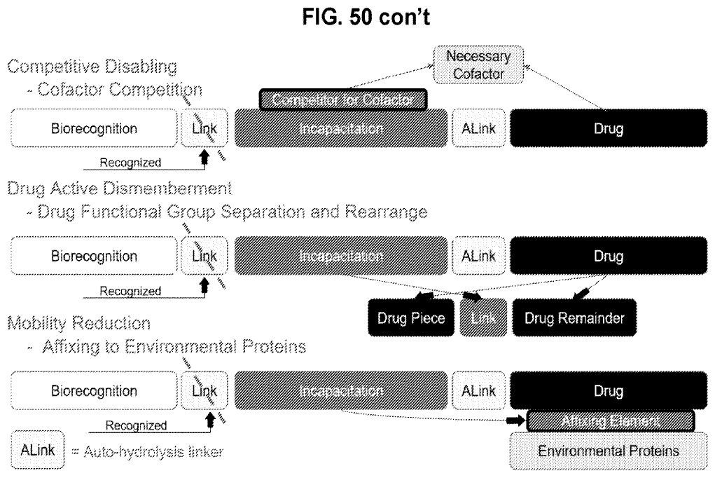

An incapacitation component is a component that inactivates or incapacitates the drug component in response to the biorecognition component being recognized and reacting with the indicator enzyme. FIG. 50 shows the 5 different modes for the incapacitation mechanism. For simplicity in the specific depictions, the biorecognition element is shown just for an arbitrary peptide with the reaction being cleavage by the indicator protease. Also some sample drug components are shown. Other biorecognition elements and drug components may also be compatible with the mechanisms described herein. In addition, the depicted examples all use, but are not limited to the use of, a subcomponent which is a 2- or 4-substituted benzyl carbamate subcomponent covalent bound to the recognition peptide through a dipeptide linker. Upon the recognition and substrate cleavage, this subcomponent undergoes 1,4- or 1,6-methide elimination to then trigger the downstream mechanisms in each mode. For simplicity in description, we refer to it later as the release trigger of the incapacitation component. This is just one implementation choice and may be replaced with others to achieve the same scope of the modes.

In one embodiment (Mode 1), the incapacitation mechanism is decoy presentation. Upon enzymatic recognition and reaction (cleavage, bond/electron transfer or otherwise) of the biorecognition element, the incapacitation mechanism presents a more favorable target for the drug component than the actual biological target. The drug then chooses to preferentially interact with that target and thus neutralizes itself leaving behind inert byproducts.

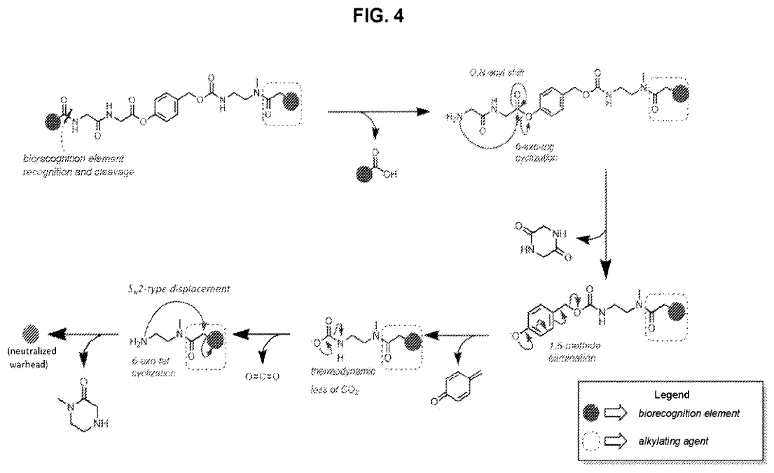

FIG. 4 shows one specific example of decoy presentation. FIG. 4 shows that a drug (an alkylating agent) is inactivated through targeting itself for alkylation upon biorecognition and cleavage of the recognition peptide. The drug, for example, includes alkyl chlorides, vinyl sulfones, acrylates, epoxides, etc. The alkylating agent in specific is depicted as an α-chloracetamide, but in other embodiments might be any α-halocarbonyl, acrylate, vinylsulfone, epoxycarbonyl, diazirine, diazo, or other moieties that can form covalent bonds with biological targets.

After the release trigger is eliminated, the remaining subcomponent is a more favorable reaction substrate for the drug component (e.g., an alkylating agent) than the drug's biological target (e.g., DNA) and forms a six-member ring, which becomes no longer active to a biological target. As an example, this remaining subcomponent may comprise a primary or secondary amine, thiol, or other similar heteroatom with potentially nucleophilic character, that is covalently acylated through a carbonate, carbamate, thiocarbonate, thiocarbamate, sulfate, or phosphate linkage to a release trigger; the release trigger is covalently positioned five, six, or seven atoms separated from the drug component.

In one embodiment (Mode 2), the incapacitation mechanism is competitive disabling. Upon biorecognition and cleavage of the recognition element, the incapacitation mechanism will present a subcomponent that will either (i) directly attack, disable or interfere with the drug, or (ii) prevent or compete with the drug-target interaction. In this case the subcomponent is not an alternate target or bait as in the first mode but a direct and active drug competitor or attacker.

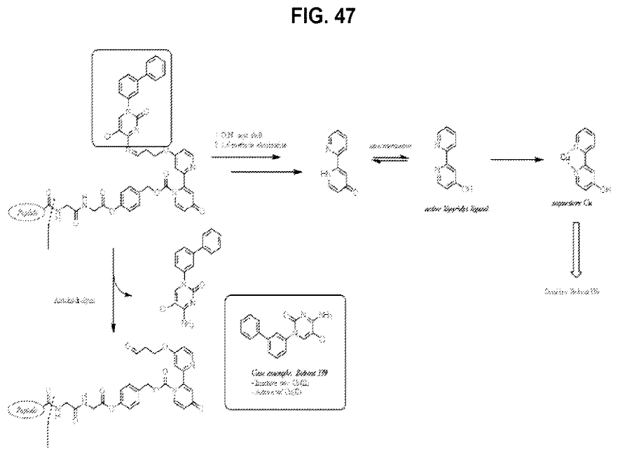

FIG. 47 shows one specific example of competitive disabling. In this case, the drug is naturally released from the compound through auto hydrolysis and takes effect, however, when the peptide is recognized and cleaved, the release trigger elimination also frees a drug competitor. In this specific case, the drug is a metal co-factor dependent inhibitor (Bobcat 339 for drug TET1 and TET2) and its competitor a metal sequestering agent which sequesters the copper needed by the inhibitor to function.

In one embodiment (Mode 3), the incapacitation mechanism is drug component dismemberment. Upon biorecognition and reaction of the biorecognition element, the incapacitation mechanism separates subcomponents that comprise the otherwise active drug component, rendering it inert.

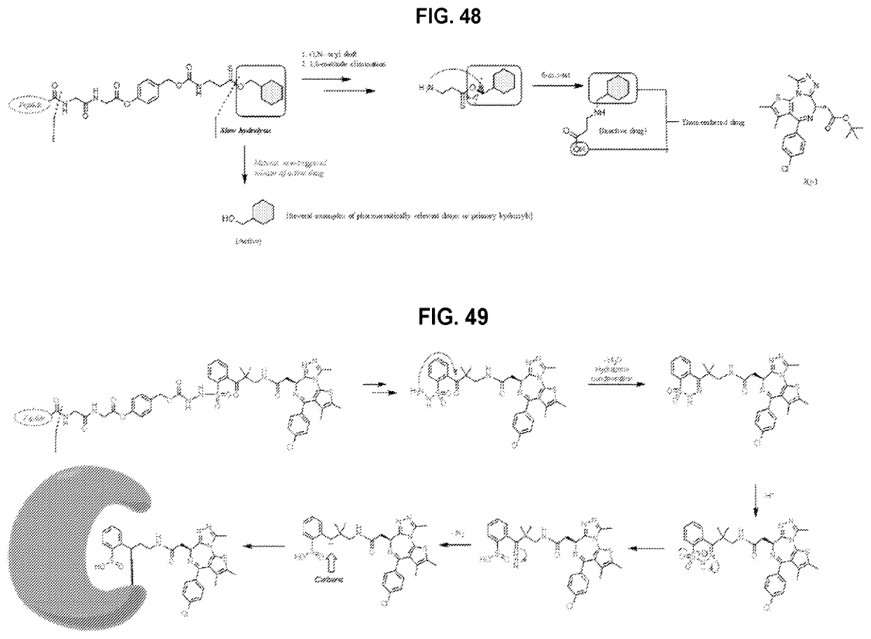

FIG. 48 shows one specific example of drug component dismemberment. Here, again the drug, which in this embodiment can be any primary hydroxyl-containing small bioactive molecule, auto hydrolyses to be active though slowly. However, the fast release trigger elimination when the peptide is cleaved triggers a 6-exo-tet reaction to also cleave and rearrange a hydroxyl group, a common generic element of many drugs, from the rest of the drug component, breaking it and rendering it inert.

In one embodiment (Mode 4), the incapacitation mechanism is increasing clearability. Upon biorecognition and reaction of the biorecognition element, the incapacitation mechanism makes the drug component more apt for clearance by the cell, tissue, or body, for example, by changing a key property such as charge, lipophilicity, or solubility.

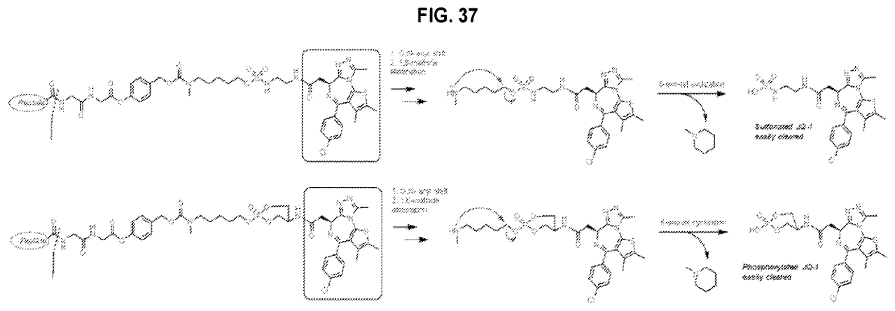

FIG. 37 shows one specific example of increasing clearability. In this example, biorecognition and cleavage of the biorecognition peptide the release trigger elimination leads to a self-cyclization of the proximal amine to displace an internal sulfonate, sulfamate, or sulfate, which is distally covalently linked to the otherwise active JQ-1 drug component. This forms a poorly absorbable sulfonated byproduct that is easily cleared through the bloodstream.

In one embodiment (Mode 5), the incapacitation mechanism is mobility reduction. Upon recognition and reaction of the biorecognition element, the incapacitation mechanism will alter mobility of the drug to make it slow moving or completely immobile.

FIG. 49 shows one specific example of mobility reduction. In this example, after recognition and cleavage of the recognition peptide, the release trigger elimination leads to formation of a benzylic diazo, which upon extrusion of nitrogen provides a carbene, which could react via C—H insertion into its surrounding molecules in the environment via a Shapiro/Bamford Stevens like Tosylhydrazone elimination (note the addition of adjacent germinal methyls to prevent elimination as in a Bamford). This links the drug component to those surrounding molecules thus preventing it from moving towards the biological target.

In some embodiments, the incapacitation component has a set of linkers that comprises a release trigger covalently linked to the biorecognition element, wherein the release trigger is cleaved from the biorecognition element upon substrate recognition and reaction with the enzyme.

The release trigger can comprise any suitable functional group that can participate in an elimination reaction. In some embodiments, the release trigger comprises a 2- or 4-substituted benzyl carbamate. In some embodiments, the release trigger undergoes 1,4- or 1,6-methide elimination upon substrate recognition and reaction with the enzyme.

The linkers of the incapacitation component are useful for triggering the incapacitation mechanism by degrading the auto-regulation compound into inert by-products. In some embodiments, the linkers degrade into biologically inert components upon substrate recognition and reaction with the enzyme.

In some embodiments, the incapacitation component comprises a linker of Formula (I):

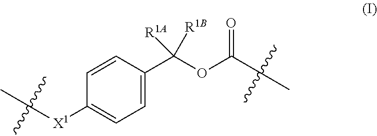

wherein: X¹ is O or NH; and R^{1A} and R^{1B} are each independently H or C₁₋₆ alkyl.

In some embodiments, the incapacitation component comprises a linker of Formula (I):

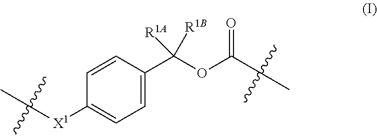

wherein: X¹ is O or NH; X² is O or NH; and R^{1A}, R^{1B}, R^{2A}, and R^{2B} are each independently H or C₁₋₆ alkyl.

X¹ can be any suitable electronegative atom. In some embodiments, X¹ is O or NH. In some embodiments, X¹ is O. In some embodiments, X¹ is NH.

X² can be any suitable electronegative atom. In some embodiments, X² is O or NH. In some embodiments, X² is O. In some embodiments, X² is NH.

R^{1A} can be any suitable functional group. In some embodiments, R^{1A} is H or C₁₋₆ alkyl. In some embodiments, R^{1A} is H or C₁₋₃ alkyl. In some embodiments, R^{1A} is H. In some embodiments, R^{1A} is C₁₋₃ alkyl. In some embodiments, R^{1A} is methyl or ethyl. In some embodiments, R^{1A} is methyl. In some embodiments, R^{1A} is ethyl.

R^{1B} can be any suitable functional group. In some embodiments, R^{1B} is H or C₁₋₆ alkyl. In some embodiments, R^{1B} is H or C₁₋₃ alkyl. In some embodiments, R^{1B} is H. In some embodiments, R^{1B} is C₁₋₃ alkyl. In some embodiments, R^{1B} is methyl or ethyl. In some embodiments, R^{1B} is methyl. In some embodiments, R^{1B} is ethyl. R^{2B} can be any suitable

R^{2A} can be any suitable functional group. In some embodiments, R^{2A} is H or C₁₋₆ alkyl. In some embodiments, R^{2A} is H or C₁₋₃ alkyl. In some embodiments, R^{2A} is H. In some embodiments, R^{2A} is C₁₋₃ alkyl. In some embodiments, R^{2A} is methyl or ethyl. In some embodiments, R^{2A} is methyl. In some embodiments, R^{2A} is ethyl.

R^{2B} can be any suitable functional group. In some embodiments, R^{2B} is H or C₁₋₆ alkyl. In some embodiments, R^{2B} is H or C₁₋₃ alkyl. In some embodiments, R^{2B} is H. In some embodiments, R^{2B} is C₁₋₃ alkyl. In some embodiments, R^{2B} is methyl or ethyl. In some embodiments, R^{2B} is methyl. In some embodiments, R^{2B} is ethyl.

In some embodiments, R^{1A} and R^{1B} are each independently H or C₁₋₃ alkyl. In some embodiments, R^{1A} and R^{1B} are H. In some embodiments, R^{1A} and R^{1B} is methyl. In some embodiments, R^{2A} and R^{2B} are each independently H or C₁₋₃ alkyl. In some embodiments, R^{2A} and R^{2B} are H. In some embodiments, R^{2A} and R^{2B} are methyl.

The incapacitation mechanism can either inactivate or incapacitate the drug component, and is triggered upon cleavage of a covalent bond between the biorecognition component and the incapacitation component. In some embodiments, the incapacitation mechanism is decoy presentation, competitive disabling, drug component dismemberment, increased clearability, or mobility reduction.

In some embodiments, the incapacitation mechanism is decoy presentation. Decoy presentation uses the mechanism of presenting a more favorable target that the drug component preferentially interacts with the drug component instead of the biological target upon cleavage of the biorecognition component. The incapacitation component can interact with the drug component in order to incapacitate the drug. When the drug is an alkylating agent, the cleavage of the biorecognition component exposes a site on the incapacitation component that is more favorable for alkylation. When the incapacitation component involves an elimination of one of the subcomponents or linkers of the incapacitation, the remaining subcomponent of the incapacitation component covalently linked to the drug component form a six-member ring with the alkylating agent, yielding an inert byproduct instead of interacting with the biological target. In some embodiments, the remaining subcomponent can be, but is not limited to a primary or secondary amine, thiol, or other similar heteroatom with potentially nucleophilic character, that is covalently acrylate through a carbonate, carbamate, thiocarbonate, thiocarbamate, sulfate, or phosphate linkage to the eliminated component. In some embodiments, upon substrate biorecognition and reaction with the enzyme, the incapacitation component is cleaved from the biorecognition component and interacts with the drug component.

In some embodiments, upon substrate recognition and reaction with the enzyme, the incapacitation mechanism exposes or provides a decoy element and the drug component interacts with the decoy instead of the biological target. In some embodiments, the incapacitation component further comprises a primary amine, a secondary amine, or a thiol.

In some embodiments, the decoy element is tethered to the therapeutic agent of the drug component after the incapacitation mechanism is engaged, and offers a more favorable target. The decoy element can be a more favorable target due to the intramolecular interaction with the drug component being favored over the intermolecular interaction with the biological target.

In some embodiments, the incapacitation mechanism is competitive disabling. Competitive disabling uses the mechanism of presenting a subcomponent that will either (i) directly attack, disable, or interfere with the drug component, or (ii) prevent or compete with the drug-target interaction upon recognition and reaction of the biorecognition component. The subcomponent is a direct and active drug competitor or attacker. In some embodiments, the incapacitation component further comprises a moiety that will directly compete with the drug component to interact with the drug component's target. In some embodiments, the drug component uses a cofactor to act on the target. In some embodiments, the competitor steasl or scavenges the cofactor away form the use by the drug active. In some embodiments, the co-factor is a metal, and the competitor is a metal sequestering agent. In some embodiments, the metal is copper.

In some embodiments, the drug is a conjugate of two active therapeutic agents that oppose each other in function. The conjugation of the opposing drug elements can enable the drug action of one, the primary, over the other, the secondary. The reaction of the biorecognition component enables the secondary to take effect to counter or inhibit the action of the primary. The opposing drug elements can be an inhibitor and an activator reaction of the biorecognition component. When the primary of the inhibitor/activator pair acts at one site but reaction of the biorecognition component releases the secondary to compete at the site or to provide a counter effect at an allosteric site.

In some embodiments, the incapacitation component further comprises a moiety that will directly compete with the therapeutic agent of the drug component. The moiety can directly compete with the therapeutic agent in terms of biological effect to counter or lessen the therapeutic agent. In some embodiments, the moiety can have the same or a different target than the drug component. In some embodiments, the moiety has the same target than the drug component. In some embodiments, the moiety has a different target than the drug component.

In some embodiments, competition occurs between linked or conjugated therapeutic agents with an opposite effect. In a non-limiting example, one therapeutic agent can be an inhibitor, and the other moiety can be an activator, or vice versa. In some embodiments, the moiety is a second therapeutic agent, and competition occurs between linked or conjugated therapeutic agents with an opposite effect. In some embodiments, wherein the therapeutic agent of the drug component is dominant in effect over the moiety before the incapacitation mechanism is engaged, but after the incapacitation mechanism is engaged, the moiety is able to better compete and incapacitate the effect of the therapeutic agent. In some embodiments, the incapacitation mechanism releases the moiety from the therapeutic agent of the drug component so that it can act independently. The release of the moiety such that it can act independently allows it to increase effectiveness.

In some embodiments, the incapacitation component further comprises a moiety that will directly compete with the therapeutic agent for a cofactor necessary for the therapeutic agent. In some embodiments, the moiety is a proteasome activator or proteasome inhibitor. In some embodiments, the moiety is Bobcat339.

In some embodiments, the incapacitation mechanism is drug component dismemberment. In some embodiments, upon substrate recognition and reaction with the enzyme, the incapacitation component interacts with the drug component to separate it into drug subcomponents.

In some embodiments, the drug component has a hydroxyl group and the recognition and reaction of the biorecognition component executes the dismemberment through a 6-exo-tet reaction to cleave and rearrange a hydroxyl group from the rest of the drug component, breaking it and rendering it inert. In some embodiments, the drug component has a localizing subcomponent and the recognition and reaction of the biorecognition element executes the dismemberment by severing the link between the localizing subcomponent and thus preventing their localization of the therapeutic agent to the biological target. In some embodiments, the drug component comprises PROTAC, and the subcomponents separate into the E3 ligase substrate and the substrate that binds the target protein. In some embodiments, the therapeutic agent of the drug component has a glycoside group and the recognition and reaction of the biorecognition component executes the dismemberment through a 6-exo-tet reaction to cleave the glycoside group from the rest of the drug component, rendering it inert.

In some embodiments, the drug component is a conjugate of two elements needed for the therapeutic agent's activity, and dismemberment comprises separation of the two elements. In some embodiments, the conjugate of two elements is a bifunctional molecule. In some embodiments, the bifunctional molecule is PROTAC.

In some embodiments, one of the subcomponents is a functional group of the therapeutic agent of the drug component. In some embodiments, the functional group is a hydroxyl group.

In some embodiments, the subcomponents can be reattached to each other, such that they do not enable the drug component to retain its biological functional capacity. In some embodiments, the drug component is JQ-1.

In some embodiments, the incapacitation mechanism is increasing clearability. In some embodiments, the incapacitation mechanism is increased clearability, wherein the increasing clearability is from tissue, cell, organelles, subcellular or tissue localized region, from systemic circulation, or from the body as a whole. In some embodiments, the incapacitation component engages in a self-cyclization of a proximal amine. In some embodiments, the self-cyclization results in changing the lipophilicity, solubility, or charge of the drug component, thereby resulting in an increased clearability of the drug component. In some embodiments, the increased clearability occurs through changing the lipophilicity, solubility, or charge of the drug component.

In some embodiments, the increasing clearability is engaged by self-cyclization of a proximal amine to displace an internal functional group of the drug component to incapacitate the drug component. In some embodiments, the internal functional group is sulfamate or sulfate, which is distally covalently linked to the drug component, thus forming a poorly absorbable sulfonated byproduct that is easily cleared by the bloodstream. In some embodiments, the self-cyclization is initiated by a release trigger.

In some embodiments, when the therapeutic agent of the drug component requires an entry functional group, the increased clearability is achieved via removal of the entry element upon recognition and reaction of the biorecognition component, thus incapacitating the drug component by preventing uptake of the therapeutic agent and increase the clearability.

In some embodiments, changing the lipophilicity, solubility, or charge of the drug component comprises adding or exposing a functional group tag on the drug to enhance clearance. In some embodiments, the functional group tag is a sulfonate group. In some embodiments, the functional group tag is a phosphate group. In some embodiments, the drug component is rapamycin. In some embodiments, the drug component is JQ-1.

In some embodiments, the incapacitation mechanism is mobility reduction. In some embodiments, the incapacitation mechanism is mobility reduction, wherein the mobility reduction comprises slowing or halting the mobility to the drug towards its target. In some embodiments, the mobility reduction occurs through the removal of a mobility element for traversing, entering, localizing, or targeting in the body, in organs, in tissues, in cells, organelles, or a local subcellular or extracellular region. These mobility components can also be used completely independently of the incapacitation mechanism, as described in later embodiments. In some embodiments, upon substrate recognition and reaction with the enzyme incapacitation component results in the formation of a carbene. In some embodiments, the incapacitation component further comprises the following structure:

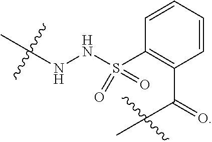 [SMILES: *NNS(=O)(=O)c1ccccc1C(*)=O]

In some embodiments, the mobility reduction is can be achieved via the formation of a benzylic diazo group, which upon extrusion of the nitrogen, provides a carbene, wherein the carbene can react via a C—H insertion into its surrounding molecules in the environment via a Shapiro/Bamford Stevens-like tosylhydrazone elimination. The covalent bond formed from the carbene reaction links the drug component to the surrounding molecules, thus incapacitating the drug since it is prevented from moving toward the biological target. In some embodiments, the benzylic diazo formation is initiated by a release trigger. In some embodiments, the mobility reduction further comprises the addition of adjacent germinal methyl groups to prevent elimination as in a Bamford reaction.

### Drug Component

A drug component suitable for the present invention includes any drug that retains efficacy after being appropriately modified for attachment to the incapacitation component in the chemical structure, and becomes inactive after the recognition and reaction of the biorecognition component engages the incapacitation mechanism. In one embodiment, the drug comprises an α-haloacetamide (e.g. α-choloacetamide), α-halocarbonyl, acrylate, vinylsulfone, epoxycarbonyl, diazirine, diazo, or other moieties that can form covalent bonds with biological targets.

The drug may be small molecules, large molecules, peptide, proteins, nucleic acids, lipids or other metabolites. Small molecules are preferred drug components.

The drug may have the activity of cytotoxicity, inhibition, activation, sequestration, biosynthesis, anabolic activity, or catabolic activity.

In some embodiments, the drug component retains efficacy when covalently bonded to the incapacitation component, wherein the incapacitation component is covalently bonded to the biorecognition component. In some embodiments, the drug component retains efficacy when the incapacitation mechanism is not triggered, wherein the incapacitation component is covalently bonded to the biorecognition component.

In some embodiments, the drug component is a small molecule, macromolecule, peptide, protein, nucleic acid, lipid, metabolite, antibody, or hormone. In some embodiments, the drug component is a small molecule, macromolecule, peptide, protein, nucleic acid, lipid or metabolite. In some embodiments, the drug component is a small molecule. In some embodiments, the drug component is a macromolecule. In some embodiments, the drug component is a peptide. In some embodiments the drug component is a protein. In some embodiments, the drug component is a nucleic acid. In some embodiments, the drug component is a lipid. In some embodiments, the drug component is a metabolite.

In some embodiments, the drug component is an alkylating agent. In some embodiments, the alkylating agent is an alkyl chloride, vinyl sulfone, acrylate, or epoxide. In some embodiments, the alkylating agent is α-chloracetamide, α-halocarbonyl, acrylate, vinylsulfone, epoxycarbonyl, diazirine, diazo, glufosfamide, ifosfomide, or lomustine.

In some embodiments, the drug component is an intercalating agent. In some embodiments, the intercalating agent is amonafide. In some embodiments, the drug component is an antimetabolite. In some embodiments, the antimetabolite is floxuridine, gemcitabine, or 5-fluorouracil.

In some embodiments, the drug component is a small molecule enzyme inhibitor. In some embodiments, the small molecule enzyme inhibitor inhibits TET1, TET2, or a combination thereof. In some embodiments, the small molecule enzyme inhibitor is Bobcat 339. In some embodiments, the small molecule enzyme inhibitor inhibits topoisomerase. In some embodiments, the small molecule enzyme inhibitor is amonafide, SN-38, or etoposide. In some embodiments, the drug component is a small molecule bromodomain inhibitor. In some embodiments, the small molecule bromodomain inhibitor is a BET inhibitor. In some embodiments, the small molecule bromodomain inhibitor is JQ-1. In some embodiments, the drug component is a small molecule kinase inhibitor. In some embodiments, the small molecule kinase inhibitor inhibits mTOR. In some embodiments, the small molecule kinase inhibitor is rapamycin.

In some embodiments, the drug component comprises at least two subcomponents that have different biological targets. In some embodiments, the drug component is PROTAC. In some embodiments, PROTAC comprises a BRD4 ligand. In some embodiments, the BRD4 ligand is JQ-1. In some embodiments, PROTAC comprises a TrkA ligand. In some embodiments, TrkA ligand has the peptide sequence IENPQYFSDA.

In some embodiments, the drug component is glycosolated. In some embodiments, the drug component is etoposide, ertyhromycin, proscillardin A, ivermectin or digitoxin.

In some embodiments, the drug component is a proteasome inhibitor. In some embodiments, the proteasome inhibitor is bortezomib, ixazomib, or MG132. In some embodiments, the drug component is a proteasome activator. In some embodiments, the proteasome activator is a peptide. In some embodiments, the proteasome activator is RRRRPPP.

In some embodiments, the drug component is an imaging agent. In some embodiments, the imaging agent is fluorescein.

In some embodiments, the compound is:

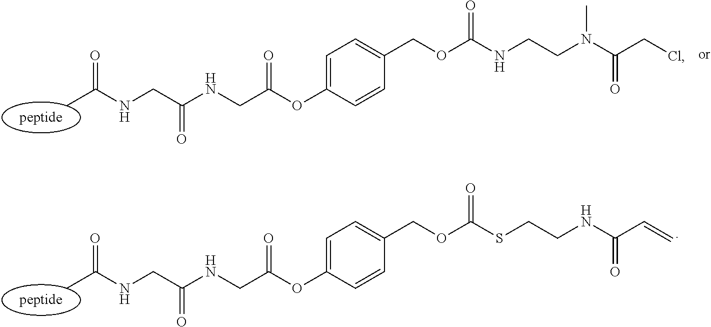 [SMILES: CC(=O)NCC(=O)NCC(=O)Oc1ccc(COC(=O)NCCN(C)C(=O)CCl)cc1 ; "or" ; C=CC(=O)NCCSC(=O)OCc1ccc(OC(=O)CNC(=O)CNC(C)=O)cc1]

### Additional Components

In one embodiment, the chemical structure of the present invention further includes an additional entry component which enables or facilitates entry into the cell, tissue or system but does not interfere with the biorecognition, drug activity or incapacitation capability of the chemical structure. Examples of the entry components include, but are not limited to, cell entry peptides, fusogenic, endocytic, or other such entry proteins, antibodies, or other component that provides cell membrane interaction and penetration. For example, the entry component may be a poly-arginine peptide. For example, the entry component comprises a peptide sequence of RRRRNRTRRNRRRVR, RRRRRRRRRRRR, PPPPPPPPPRRRRRRRW, GRKKRRQRRRPPQ, RQIKIWFQNRRMKWKK, RRRRRRRRR. or RRRRRRRR.

In some embodiments, the auto-regulating compound further comprises a cell entry component. In some embodiments, the cell entry component facilitates entry into the cell tissue or system, but does not interfere with the biorecognition component, the incapacitation component, or the drug component. In some embodiments, the cell entry component is a cell entry peptide, a fusogenic, an endocytic, or an antibody. In some embodiments, the cell entry component comprises poly-arginine peptide. In some embodiments, the poly-arginine peptide comprises a sequence of RRRRNRTRRNRRRVR, RRRRRRRRRRRR, PPPPPPPPPRRRRRRRW, GRKKRRQRRRPPQ, RQIKIWFQNRRMKWKK, RRRRRRRRR. or RRRRRRRR. In some embodiments,

In one embodiment, the chemical structure of the present invention further includes an additional localization component. A localization component enables or facilatates localization to a specific region within the cells, tissue or system and it does not interfere with the biorecognition, drug activity or incapacitation capability of the chemical structure. A localization component may help to locate the chemical structure to specific regions including a cell membrane, cytoskeletal elements, or organelles such as the nucleus, mitochondria, endoplasmic reticulum, ribosomes or Golgi bodies, or specific subcompartments of the aforementioned. For example, a localization component is a peptide sequence for nuclear localization.

In some embodiments, the auto-regulating compound further comprising a localization component. In some embodiments, the localization component facilitates localization to a specific region within the cell tissue or system, but does not interfere with the biorecognition component, the incapacitation component, or the drug component. In some embodiments, the localization component locates the chemical structure to specific regions including a cell membrane, cytoskeletal elements, or organelles such as the nucleus, mitochondria, endoplasmic reticulum, ribosomes or Golgi bodies. In some embodiments, the localization component is a peptide sequence for nuclear localization. In some embodiments, the peptide sequence is RRARRPRGR, PKLKRQ, RPRK, GKRKLITSEEERSPAKRGRKS, KGKKGRTQKEKKAARARSKGKN, RKRCAAGVGGGPAGCPAPGSTPLKKPRR, RKPVTAQERQREREEKRRRRQERAKEREKRRQERER, RSGGNHRRNGRGGRGGYNRRNNGYHPY, TLLLRETMNNLGVSDHAVLSRKTPQPY, or PGKMDKGEHRQERRDRPY.

In one embodiment, the chemical structure of the present invention further comprises a localization component and an entry component.

In one embodiment, the cell entry component and the localization component are combined as an amino acid sequence, for example, RRARRPRGR, PKLKRQ, RPRK, GKRKLITSEEERSPAKRGRKS, KGKKGRTQKEKKAARARSKGKN, RKRCAAGVGGGPAGCPAPGSTPLKKPRR, RKPVTAQERQREREEKRRRRQERAKEREKRRQERER, RSGGNHRRNGRGGRGGYNRRNNGYHPY, TLLLRETMNNLGVSDHAVLSRKTPQPY, or PGKMDKGEHRQERRDRPY.

In some embodiments, these aforementioned additional elements can also be a part of the incapacitation mechanism. In some embodiments, the additional elements are not separate and part of the incapacitation mechanism of mode 5. In some embodiments, the additional elements are mobility elements which can be removed to reduce the mobility of the drug to incapacitate it.

### Methods of Treatment

The chemical structure of the present invention is suitable to treat different diseases, this is because enzymatic processes are fundamental, and their failure or general impairment is a feature of most diseases.

The chemical structure of the present invention is suitable to be administered to cells or tissues of a subject for treating a disease where there exists a target enzyme which (1) decreases in its presence or its activity as the disease progresses, and (2) has a specific substrate recognized by that enzyme. For example, cancer is one example of diseases suitable to be treated by the present invention. For example, cancers that can be distinguished by reduction of the prostasin marker enzyme include breast, colorectal, squamous cell carcinoma and prostate.

In some embodiments, is a method of treating a disease or disorder, the method comprising administering to the subject an auto-regulating compound described herein, or a pharmaceutically acceptable salt or solvate thereof.

In some embodiments, is a method of tuning the amount of a therapeutic agent for the treatment of a disease or disorder in a subject, wherein the subject is administered a therapeutically effective amount of the therapeutic agent when the disease or disorder is progressing, and the subject is not administered a therapeutically effect amount of the therapeutic agent when the disease or disorder is mitigated, the method comprising: administering to the subject a auto-regulating compound, wherein the auto-regulating compound comprises (i) a biorecognition component comprising a substrate; (ii) an incapacitation component; and (iii) a drug component comprising the therapeutic agent, wherein the incapacitation component is covalently linked to the biorecognition component and the drug component; wherein the auto-regulating compound has an incapacitation mechanism, wherein when the disease or disorder is progressing, the auto-regulation compound is active; wherein when the disease or disorder is mitigated, the auto-regulation compound is incapacitated via an incapacitation mechanism comprising: (a) reacting the biorecognition component with an enzyme, wherein the enzyme is absent or in low concentration during disease or disorder progression, and the enzyme is in higher concentration during disease or disorder mitigation; (b) cleaving of a covalent bond between the biorecognition component and the incapacitation component, thereby triggering the incapacitation mechanism; and (c) incapacitating the drug component with the incapacitation component which is no longer covalently linked to the biorecognition component; thereby tuning the amount of the therapeutic agent for the treatment of a disease or disorder.

In some embodiments, is a method of tuning the amount of a therapeutic agent for the treatment of a disease or disorder in a subject, wherein the subject is administered a therapeutically effective amount of the therapeutic agent when the disease or disorder is present or progressing, and the subject is not administered a therapeutically effect amount of the therapeutic agent when the disease or disorder is mitigated or in areas where there are healthy cells, tissues, or organs, the method comprising: administering to the subject an auto-regulating compound, wherein the auto-regulating compound comprises (i) a biorecognition component comprising a substrate; (ii) an incapacitation component; and (iii) a drug component comprising the therapeutic agent, wherein the incapacitation component is covalently linked to the biorecognition component and the drug component; wherein the auto-regulating compound has an incapacitation mechanism, wherein when the disease or disorder is progressing, the auto-regulation compound carries out its biological function; wherein when the disease or disorder is mitigated or where there are healthy cells, tissues, or organs, the auto-regulation compound is incapacitated via an incapacitation mechanism comprising: (a) reacting the biorecognition component with an enzyme, wherein the enzyme is absent or in low concentration during and in regions of disease or disorder presence or progression, and the enzyme is in higher concentration during disease or disorder mitigation or where there are healthy cells, tissues, or organs; (b) cleaving of a covalent bond between the biorecognition component and the incapacitation component, thereby triggering the incapacitation mechanism; and (c) incapacitating the drug component with the incapacitation component which is no longer covalently linked to the biorecognition component; thereby tuning the amount of the therapeutic agent for the treatment of a disease or disorder, and reducing side and off target effects.

The compounds useful for the methods described herein are the auto-regulating compounds described herein.

In some embodiments, the disease or disorder has a target enzyme which (1) decreases in presence or activity as the disease progresses, and (2) has a specific substrate recognized by the target enzyme.

In some embodiments, the disease or disorder is cancer. In some embodiments, the cancer has reduction of prostasin enzyme. In some embodiments, the cancer is breast cancer, colorectal cancer, squamous cell carcinoma, or prostate cancer.

In some embodiments, the disease or disorder is protein aggregation disease. In some embodiments, the disease is a neurodegenerative disease, Alzheimer's, Parkinson's, amyotrophic lateral sclerosis, dementia with Lewy bodies, frontotemporal dementia, or Huntington's disease.

In some embodiments is a kit for tuning the amount of a therapeutic agent for the treatment of a disease or disorder, the kit comprising: a pharmaceutical composition comprising the auto-regulating compound described herein and a pharmaceutically acceptable excipient; and an instruction manual for the usage of the auto-regulating compound.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

As used in the specification and claims, the term “incapacitate” refers to preventing or disabling the drug active, therapeutic agent, or drug component from functioning in its normal way to trigger a biological response and/or treatment. Incapacitate includes the term inactivation, but can also stop or block the drug component, drug active, or therapeutic agent from performing its biological function, for example via competition or by increasing the clearance.

As used in the specification and claims, the terms “self-regulate,” “auto-regulate,” “adaptive,” and “negative feedback,” are used interchangeably to refer to some function of the output of a mechanism, wherein the mechanism is fed back in a manner that tends to reduce the the activity of the compounds described herein, which can be caused by changes in the biological environment by other disturbance.

As used in the specification and claims, the terms “therapeutic agent” and “drug active” are used interchangeably to describe biologically active compound capable of treating at least one disease state or condition.

As used in the specification and claims, the term “small molecule” refers to compounds that are understood by one of skill in the art, for example to have a molecular weight of less than about 1000 Daltons. The small molecules can be organic compounds which are therapeutic agents that can regulate a biological process. In a non-limiting example, many chemotherapeutic agents are small molecules.

As used in the specification and claims, the term “macromolecule” refers to large compounds that are understood by one of skill in the art, typically in excess of about 1000 Daltons. The macromolecules can be useful in biological processes, with the most common macromolecules including biopolymers such as nucleic acids, proteins, and carbohydrates, as well as lipids, macrocycles, and nanogels.

As used in the specification and claims, the term “metabolite” refers to an end product or an intermediate from metabolic processes, as understood by one of skill in the art. Metabolites typically have various biological functions such as signaling, stimulatory and inhibitory effects on enzymes, catalytic activity such as functioning as cofactor to an enzyme, and interactions with other organisms. Primary metabolites are involved in normal growth, development, and reproduction. Secondary metabolites are not directly involved in the aforementioned processes, but can be indirectly involved and have an ecological function.

The section headings used herein are for organizational purposes only and are not to be construed as limiting the subject matter described.

### EXAMPLES

The following examples further illustrate the present invention. The examples are intended merely to be illustrative of the present invention and are not to be construed as being limiting.

### General Description

Indication Enzyme: Prostasin (and Matriptase collaborative enzyme)
Functional Role: Sodium Transport Channel: ENaC processing, Epithelial Junction Protein Processing, General Epithelial Identity Regulator
Use Case: Breast Cancer (Adenocarcenoma)
Pathological Trend: Diminished enzyme levels in a variety of tumor cells and even lower levels after metastasis
Test Tissue: Breast Epithelium
Recognition Sequence: Peptide Sequence QARG
Localization and Entry Elements optionally used: Peptide Sequence RRARRPRGR, acts as both.

### Example 1A. Chemical Synthesis (Specific Compound)

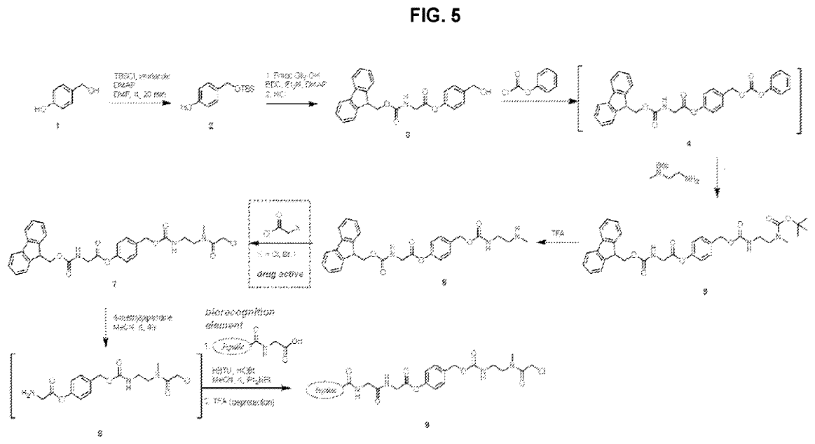

FIG. 5 shows a chemical scheme of synthesis of compound 9. The synthesis began with commercially available 4-hydroxybenzyl alcohol 1. Treatment of 4-hydroxybenzyl alcohol 1 with tert-butyl dimethyl silyl chloride, imidazole, and 4-N,N-dimethylaminopyridine (DMAP) affected selective monosilylation of 1 to afford hydroxybenzyl silyl ether 2. Subsequent Stegleich esterification of 2 with FMOC-protected glycine followed by desilylative acidic workup gave FMOC-protected hydroxyester 3. Addition of phenyl chloroformate gave efficient conversion of alcohol 3 to asymmetric carbonate 4, which upon addition of mono-(tert-butoxycarbony) protected N-methylethylenediamine gave Boc-protected carbamate 5. Removal of the Boc group was found to be efficiently conducted in a 25% v/v solution of trifluoroacetic acid (TFA) in any light chlorinated solvent (e.g. dichloroethane, DCE; chloroform, CHCl₃; dichloromethane, CH₂Cl₂) to afford amine 6. Following chromatographic purification, amine 6 could be acylated with any α-haloacetyl group with reagents such as 2-chloroacetyl chloride to afford haloacetamide 7, which we envisioned to be a key diversifiable intermediate. Treatment of 7 with 4-methylpiperidine in acetonitrile gave efficient removal of the FMOC protecting group, and we found that while the resulting primary amine 8 was too polar to purify, this could be immediately acylated with any C-terminal acid of a peptide under standard amide coupling conditions including but not limited to HBTU/HOBt, HATU/HOBt, PyBOP/HOBt, which after deprotection when and if necessary affords peptide-drug conjugate 9.

### Example 1B. Chemical Synthesis of Compounds with Generic Structures

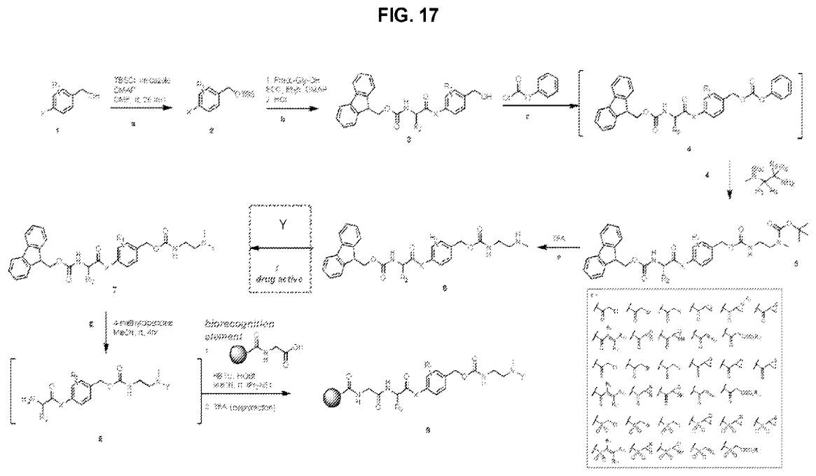

Similar to FIG. 5 and Example 1A, FIG. 17 shows a chemical scheme of synthesis of generic compounds with some varieties.

In FIG. 17 in 1 is any 4-heteroatom substituted or aryl-substituted benzyl alcohol, where ‘heteroatom’ describes any nonmetallic element other than carbon or hydrogen, including nitrogen, oxygen (as in the present embodiment), sulfur, selenium, phosphorous, or other elements not included in this list, and where ‘aryl-substituted’, denoted by R1, indicates any mono-, di-, tri-, or tetrasubstituted ortho- or para-benzyl alcohol, wherein ‘substituted’ includes any linear or branched alkyl, alkenyl, alkynyl, aryl, heteroalkyl, alkenyl, alkynyl, heteroaryl, or halogen substitution on the aromatic ring of 1 or any combinations, salts, or permutations thereof.

### Example 1C. Chemical Synthesis of Different Compound

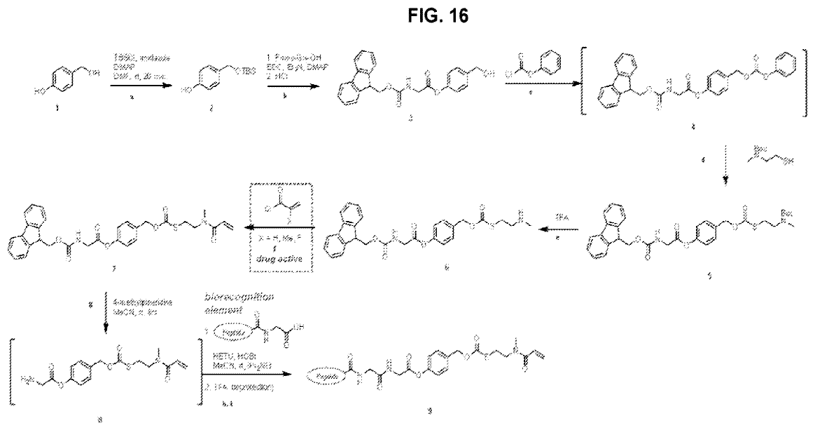

FIG. 16 shows a chemical scheme of synthesis of a different compound.

In this example, the Fmoc-Glycine acid coupling partner in step b could be substituted with any FMOC or similarly protected amino acid, where R₂ is any linear or branched alkyl, alkenyl, alkynyl, aryl, heteroalkyl, alkenyl, alkynyl, heteroaryl, or halogen substitution or any combinations, salts, or permutations thereof with any integer number of atoms from 1-100, including any stereoisomers, constitutional isomers, geometric isomers, or any combination thereof.

In yet another example embodiment, reaction step c could consist of any 1,1-disubstituted carbonyl such as phosgene, triphosgene, diphosgene, 4-nitrophenyl chloroformate, 2,2,2-trichloroethyl chloroformate, carbonyldiimidazole, phenyl chloroformate (as in the present embodiment) or any similar reagent which fit the aforementioned descriptor, including any 1,1-disubstituted thiocarbonyl equivalent reagent.

In yet another example embodiment, the monoprotected diamine or hydroxyamine in coupling step d could be monoprotected with a Boc, Fmoc, Alloc, Troc, Cbz, or other similar protecting groups, and where R3, R4, R5, and R6 are any linear or branched alkyl, alkenyl, alkynyl, aryl, heteroalkyl, heteroaryl, or halogen substitution or any combinations, salts, or permutations thereof with any integer number of atoms from 1-100, including any stereoisomers, constitutional isomers, geometric isomers, or any combination thereof.

In yet another example embodiment, the chloroacetyl moiety in step f might be substituted for any other alkylatable or acylatable or otherwise covalently modifiable drug component, including acrylates, diazo, α-haloacetates, α-halosulfonates, epoxypropanoates and the corresponding thioepoxide, aziridine, oxaziridine, or any analogous three or four membered strained system, including any alkyl, alkenyl, alkynyl, branched alkyl, alkenyl, alkynyl, heteroalkyl variation thereof.

In yet another example embodiment, the C-carboxylic acid peptide in coupling step h may be substituted with any polypeptide or polypeptidomimentic or any polymeric or monomeric set of amino acids in a linear or branched moiety of any length, including side chains containing both natural and/or unnatural/synthetic amino acid side chains, including any stereoisomers or combinations or permutations or protected variations thereof, where ‘protected’ includes any common or non-common protecting group moiety, including but not limited to trityl ethers, t-butyl ethers, silyl ethers, Boc, Fmoc, Cbz, Troc, Alloc, or other similar or dissimilar protecting groups, including any N-alkylated or N-acylated amide backbones of said peptides, and where ‘amino acids’ includes α, β, γ, δ-amino acids or any other linear or branched alkyl or heteroalkyl length separating an amine and an acid moiety, including any linear, cyclic, polycyclic, or macrocyclic variations thereof, and including any salts, ion pairs, formulations, or synthetically modified variations thereof.

### Example 2. Materials Characterization

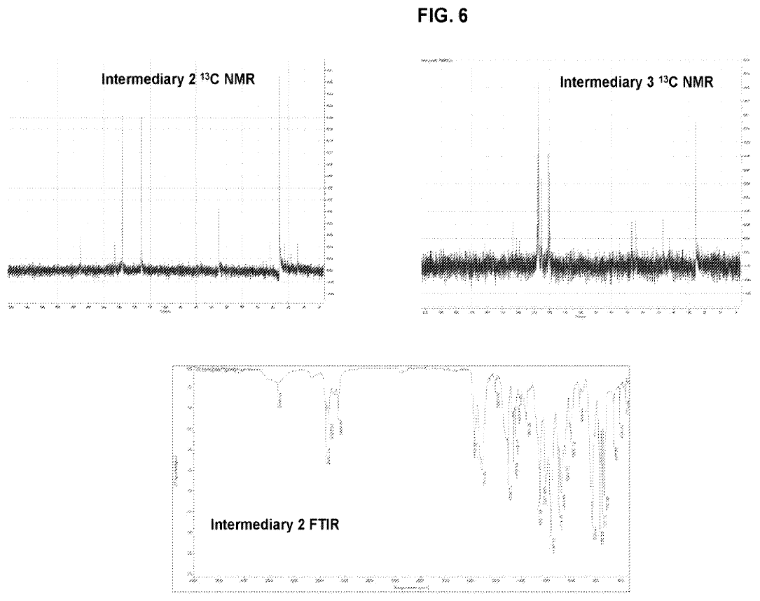

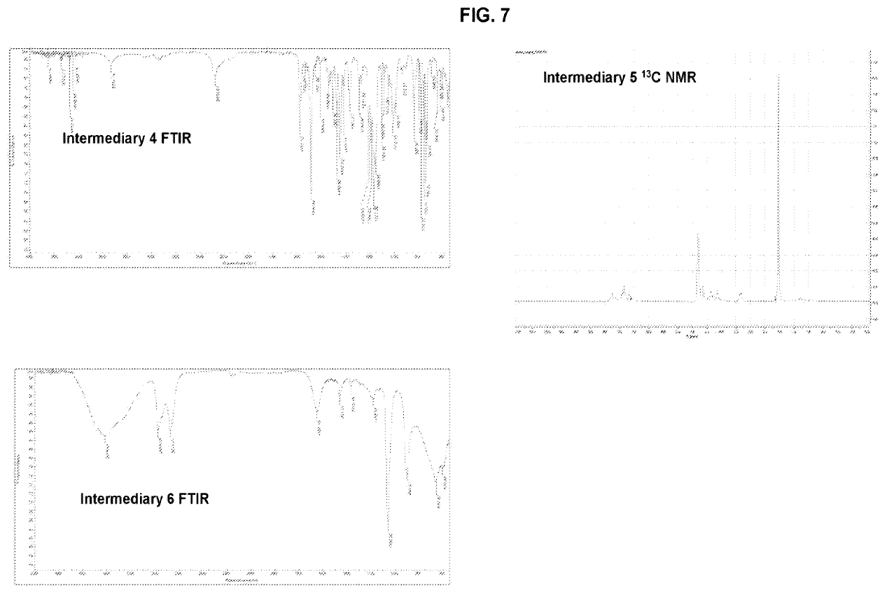

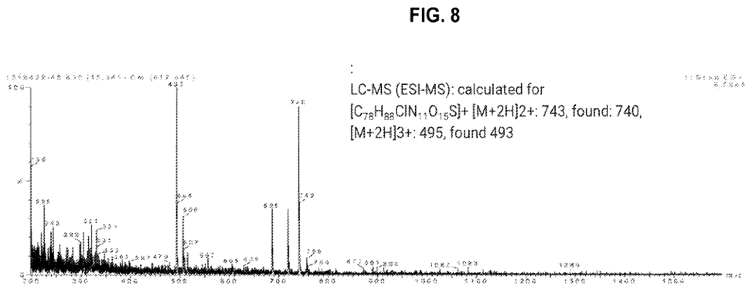

The following experiments demonstrate the characterization of Compound 9 of FIG. 5, in which peptide is QAR for prostasin. RRARRPRGRQAR was also demonstrated for cell uptake through imaging of a conjugate dancyl flurophor (data not shown here). Compound 9 was shown efficacious in these experiments. The NMR, IR, and LCMS data are provided in FIGS. 6-8.

Mass Spectrum at peak retention time of 15.34 highlights peaks at 493 and 740, triply and doubly charged species of target compound respectively.

Extracted Ion Spectrum selected for m/z 493 and 740

Peptide Sequence Validation: LCMS Validation of the correct size for recognition peptide with protecting groups (1184 Da-single charged) peptide sequence by mass spectrometry

Purified Final Product
Total Ion Chromatogram of Material with Peak Retention Time of 15:56

Mass Spectrum at peak retention time of 15.56 highlights peaks at 493 and 740, triple and double charged species of target compound respectively.

### Extracted Ion Spectrum Selected for m/z 493 and 740

### Example 3. Ex Vivo Mechanism Validation

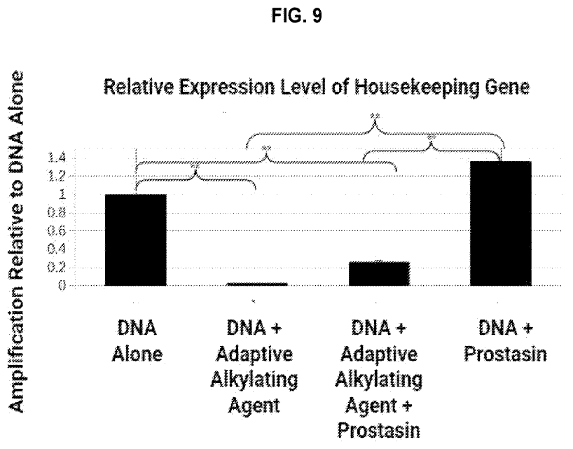

Genotoxicity Tuning. FIG. 9 shows the compound shows 98% reduction in intact standard gene (GAPDH) cDNA as measured by ability for amplification by qPCR. Recombination prostasin enzyme can be added to tune back up to 26% in this example. Enzyme alone introduces some noise but does not significantly change the amplification level. This is the analogue to synthesis and replication inhibition of DNA in vitro/vivo by the alkylating compound.

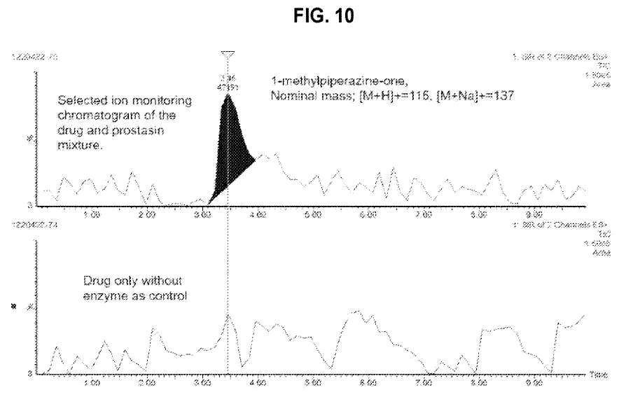

Byproduct Validation: HPLC detection of 1-methylpiperazine-one (inert byproduct of incapacitation) after incubation with recombinant prostasin enzyme in top chromatogram vs only noise levels detected without enzyme is shown in FIG. 10.

Biological Validation. Evaluation of prostasin levels on MCF10a, MCF7 and MDA-MB-231 breast epithelium cell lines is shown in FIG. 2.

Biorecognition Activity Validation. 7-Amino-4-methylcoumarin (AMC) reporter is attached to Prostasin/Matriptase Substrate (Recognition Sequence). Upon cleavage by transmembrane enzyme, AMC molecule fluoresces in the with ex:341 nm em:441 nm shown in blue. Normal line exhibits substantially blue signal compared to MCF-7 and even more so compared to MDA-MB-231, correlating to recognition and cleavage of the test substrate.

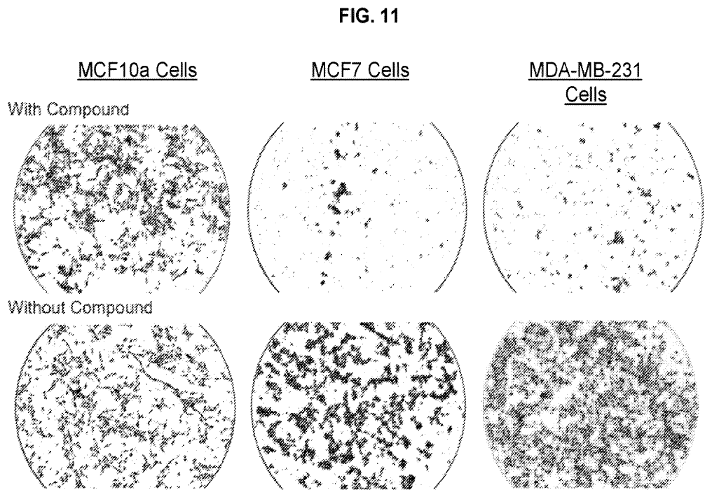

Selective Cytotoxicity Assay. All three cell lines were incubated with or without full compound at various concentrations. Displayed are brightfield images of sample wells in black and white. Widespread cell death is evident in cancer cells with compound vs without (floating debris shown in images). Healthy cells are otherwise spared by treatment and show a similar population as without compound. Shown images are from 1 hr post treatment with 1× dose compound in FIG. 11.

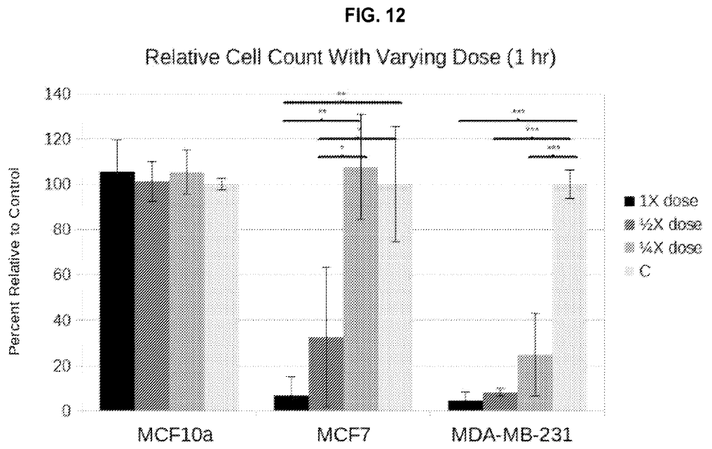

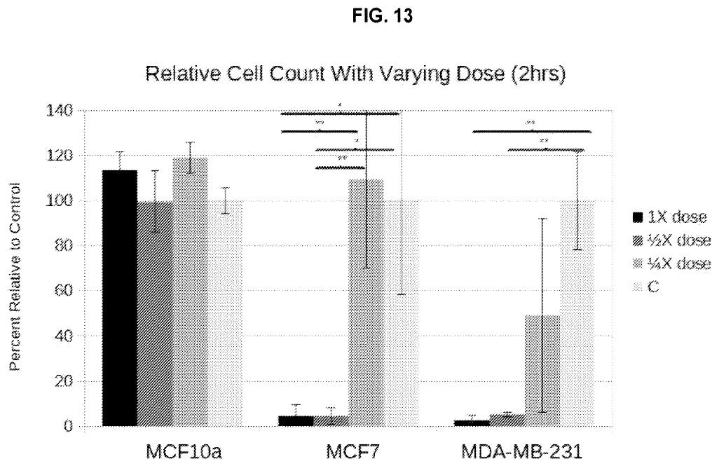

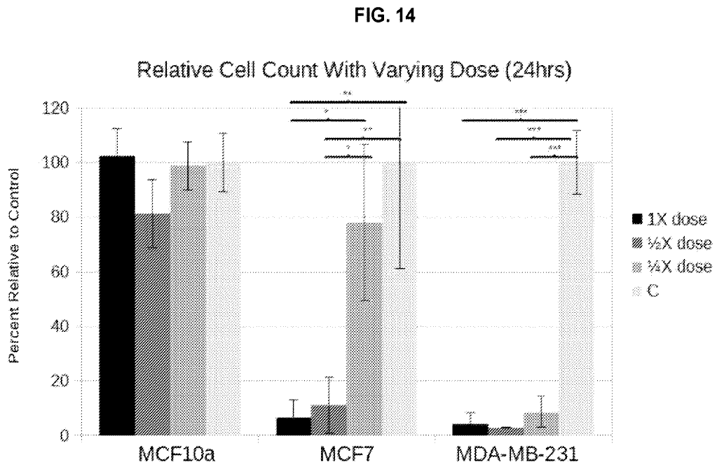

Cell Survival over Time and Dosing. FIGS. 12-14 show the cell survival over time and dosing data.

Dosing (Time and Amount): Variation Longer exposure to drug largely reinforces trend of normal cell sparing and cancer cell killing seen at 1 hr. Lower concentrations however start to become ineffective on the cancer cells as well.

### Example 4. Mode 1 Decoy Presentation with Alkylating Agent Drug Active

When the mode of decoy presentation is engaged, the incapacitation mechanism will have the drug active act on a decoy instead of the actual biological target, thus incapacitating it in its otherwise intended biological effect.

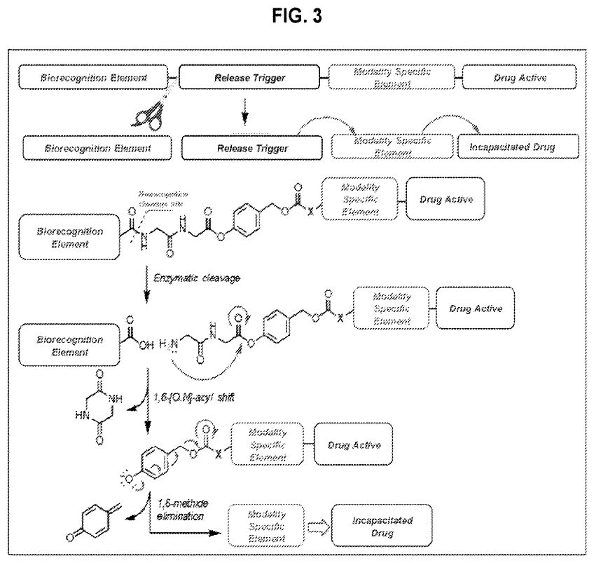

Aberrant alkylation of nucleotides (often guanine) can damage DNA and lead to shutdown or cell replication and/apoptosis. Thus alkylating agents are used in chemotherapy for clearing cancerous cells but may damage healthy cells as well. In this example, the engagement of the incapacitation mechanism releases/exposes an element (nucleophile, radical, etc.) that acts as the target of the alkylating agent through an intramolecular process rather than targeting DNA through an intermolecular process. The recognition and reaction of the biorecognition element and subsequent enzymatic cleavage of a key bond in the construct molecule triggers the exposure of a primary amine that will be the target of the alkylating agent-instead of DNA. To accomplish this design, a chemical architecture termed the release trigger depicted in FIG. 3 is used as part of the incapacitation mechanism to drive a fast intramolecular cascade and initiate the primary amine exposure. This is a diversifiable element that will be used in a number of the modalities to rapidly initiate the specific incapacitation chemistry of that modality when the biorecognition sequence is recognized and reacted upon (cleaved). This is depicted in the mechanism of FIG. 4 for an adaptive alkylating agent, specifically alkyl chloride, and the synthesis route of this compound is given in FIG. 5. Then FIGS. 6, 7 and 8 convey the steps and characterization of key intermediaries.

Demonstration of activity of the drug on isolated DNA was tested with qPCR amplification. FIG. 9 shows the effects on amplification with and without the drug and with and without the prostasin enzyme. Then in FIG. 10, incubation of the drug alone with the prostasin enzyme can be analyzed by LC/MS for the byproduct of incapacitation (the alkylation of the decoy yielding a 1-methylpiperazine-one). FIG. 11 then depicts the selective reduction in cell numbers for multiple cancer cell lines while sparing the normal cell line control. FIGS. 12, 13 and 14 then show the variation of this cell number reduction with applied dose and incubation time.

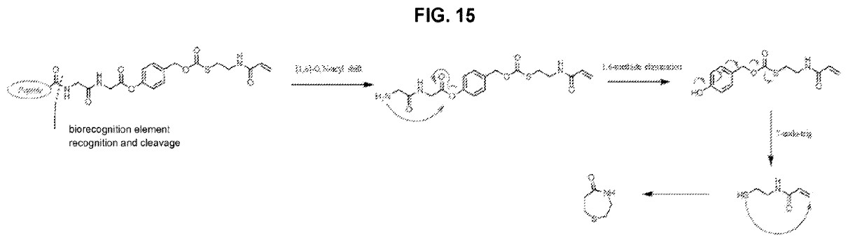

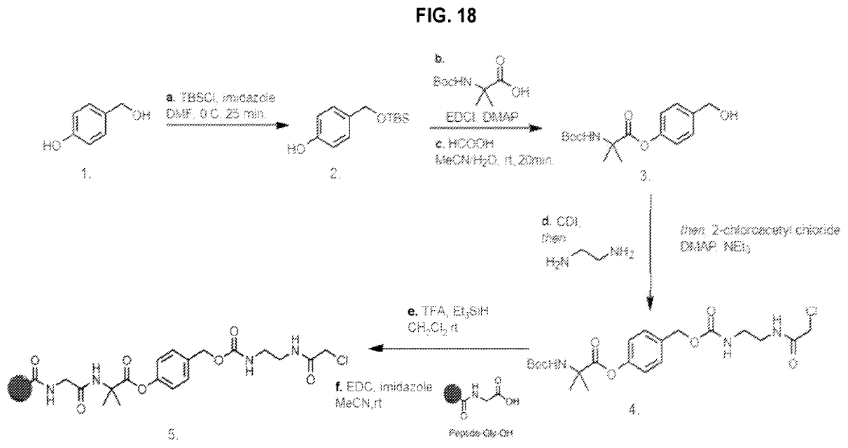

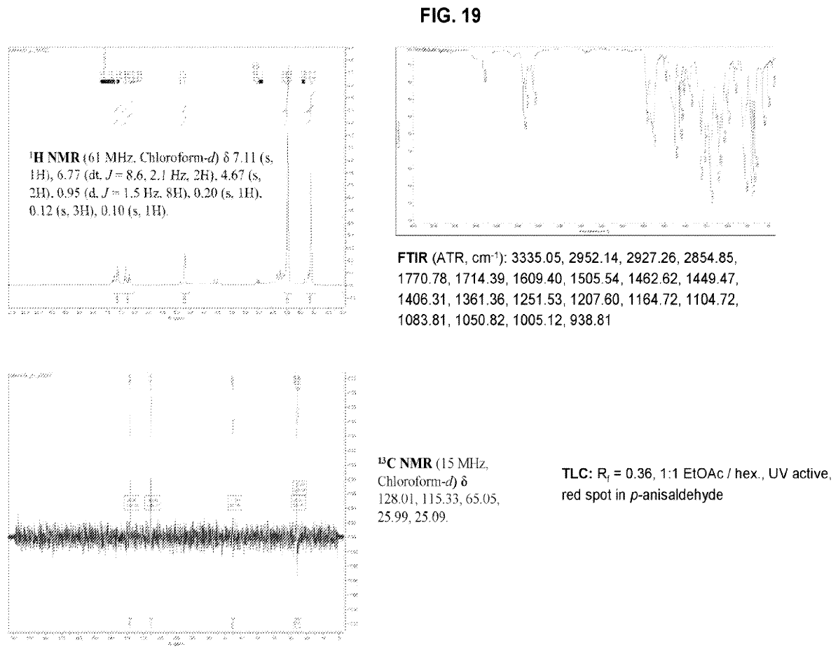

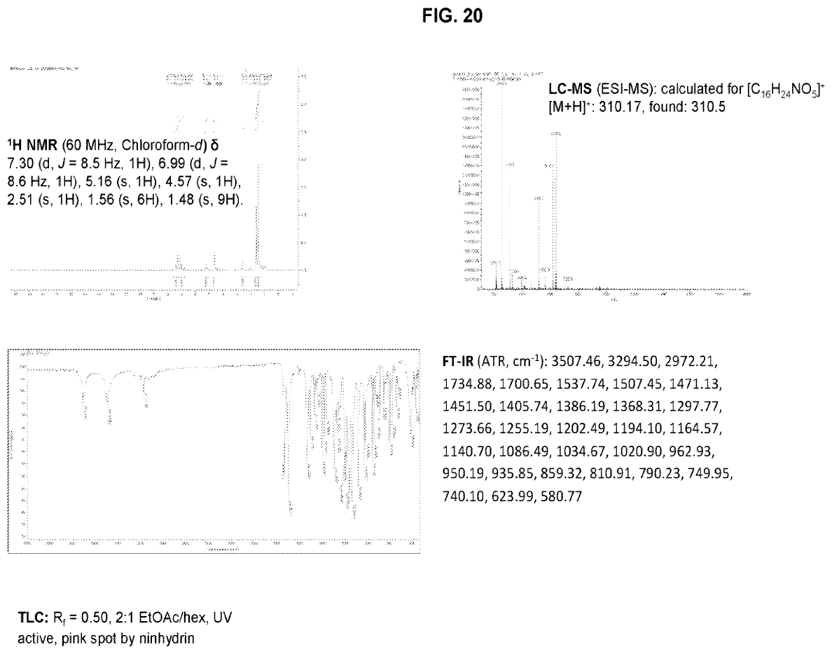

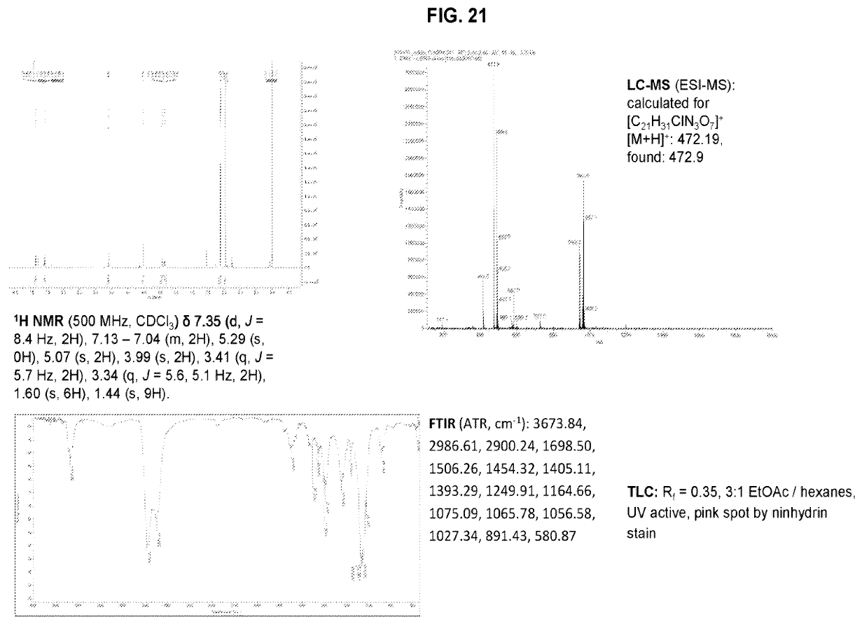

The mechanism for an alternate adaptive alkylating agent, specifically acrylate, is given in FIG. 15 and its synthesis route is given in FIG. 16. FIG. 17 then gives an even more diversifiable manifestation of these adaptive alkylating agents. FIG. 18 gives an alternate recognition trigger that can be more simply synthesized. Then FIGS. 19, 20 and 21 convey the steps and characterization of key intermediaries.

### Example 5. Mode 2 Competitive Disabling with Inhibitor/Activator Pair

When the mode of competitive disabling is engaged, a competitor compound acts to interfere with the drug active's engagement or effect on the target and/or on any necessary cofactors for the drug's function thereby incapacitating it in its otherwise intended biological effect.

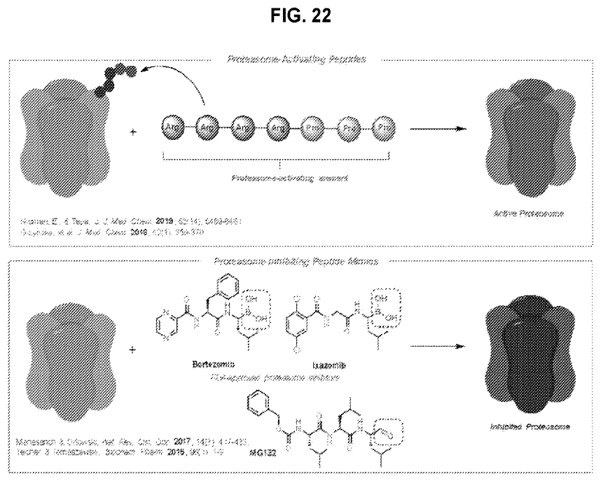

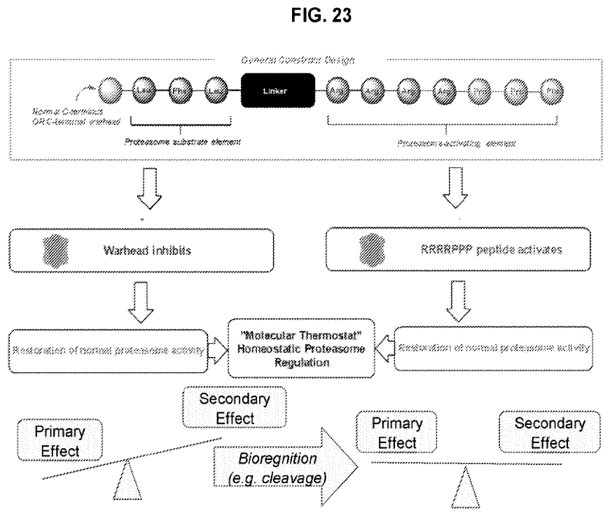

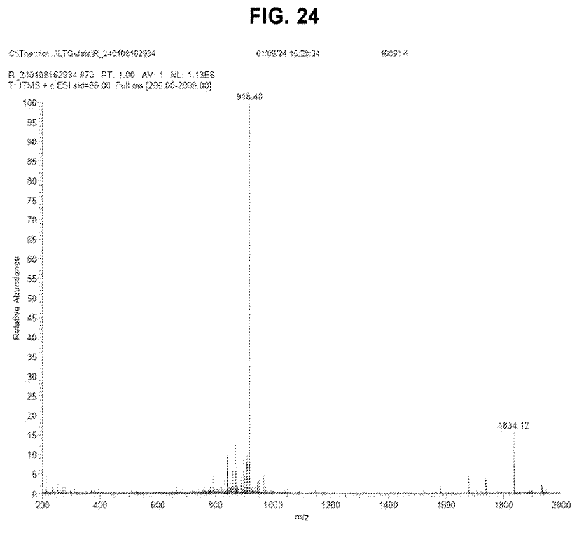

Many biological targets have drugs for both inhibition and activation. This is because the targets are implicated in different diseases, where the failure mode has an opposite polarity. For instance proteasome activity, a major protein clearance route for the cell, is elevated in fast growth and protein turnover diseases like cancer but is decreased in protein buildup and aggregation conditions like neurodegenerative disease. Thus solutions have been developed for both inhibiting and activating the proteasome as shown in FIG. 22. Yet in both cases, there is the risk of overshooting in either direction which could be detrimental. For example enhancing proteasomal activity should be enough to clear aggregates but not so much that it starts to deplete healthy levels of protein reserves. Conversely, proteasomal activity can be reduced to, for example, slow or kill fast growing cancer cells but not so much that it also prevents healthy cells from maintaining healthy levels of protein turnover. Thus the competitive disabling modality for the incapacitation mechanism can be used by coupling an activator and an inhibitor to counter each other when appropriate to prevent these forms of overshooting. In this example, the primary drug (can be either inhibitor or activator) is the more dominant in biological effect than the secondary when the two are conjugated with the adaptive logic. However, when the biorecognition sequence is recognized and acted upon, the primary drug is incapacitated (reduced) by enhancing the effect of the secondary, which has a competing and opposite effect. In this case the secondary acts as part of the incapacitation mechanism. FIG. 23 shows one such example with a peptide activator and a peptide sequence guided inhibitor, both from known literature. FIG. 24 then shows the mass spectrometry characterization of the peptide manifestation of such an adaptive drug where the engagement of the incapacitation mechanism (ie the enhancement of the effect of the secondary). In this, the recognition and cleavage of the biorecognition element severs the tether between the primary and secondary. This would allow the secondary to act freely to counter and incapacitate the effect of the primary drug. The strategy of this example can be generalized for any pair of activator and inhibitor peptides for the proteasome or any other target.

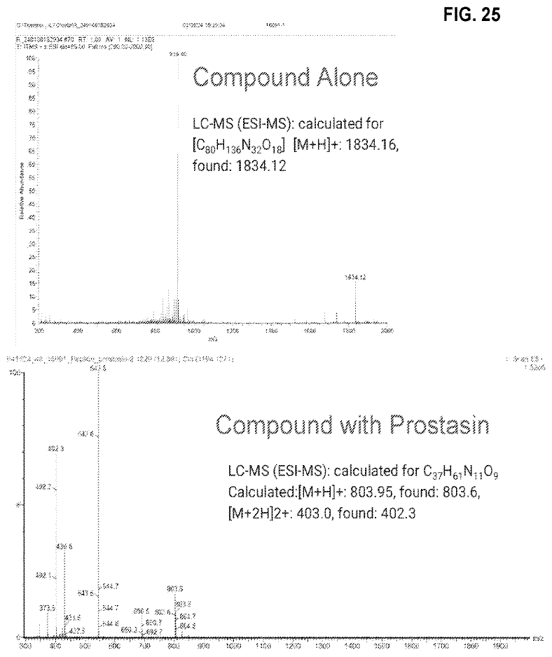

The drug could be tested in similar ways as the other compounds. Demonstration of the engagement of the incapacitation mechanism is shown by incubation of the adaptive peptide proteasome activator-inhibitor pair with the prostasin enzyme revealing the independent activator peptide in LC/MS analysis shown in FIG. 25. Additionally, cell survival and growth assessment can be done to demonstrate selective reduction of proteasome activity leading to death/slowed growth in the cancer cells while not significantly affecting the healthy cells. Demonstration of activity of the drug when the incapacitation mechanism is not engaged can be done on proteasomes isolated from cells treated with the adaptive drug using standard fluorescence based proteasomal activity detection assays-such as those commercially available from Sigma Aldrich (Cat No. MAK172).

### Example 6. Mode 3 Drug Active Dismemberment

### Peptide PROTAC Drug Active

When the mode of drug active dismemberment is engaged, the subcomponents of the drug active that are necessary for its activity are broken or separated from each other thereby incapacitating it in its otherwise intended biological effect.

Induced protein clearance can be used to remove a deleterious protein or starve a critical protein from deleterious cells. PROTAC (proteolysis targeting chimeras) drugs accomplish this by labeling the target protein and ubiquitin tag which triggers their clearance by the cellular proteasome. They do this by having two critical subcomponents—a substrate which recognizes the target protein and a substrate that can be recognized by the E3 Ligase (which initiates ubiquitination). Thus the drug active dismemberment modality for the incapacitation mechanism can be used to separate these subcomponents of the PROTAC to prevent its function.

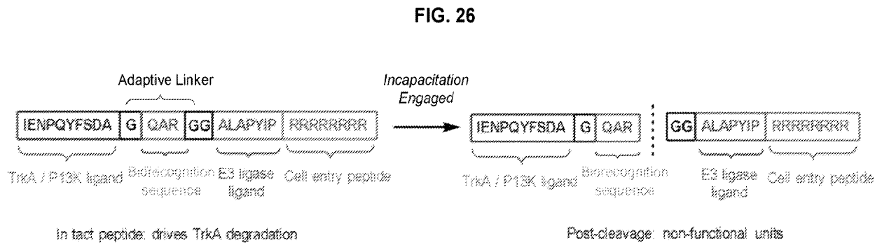

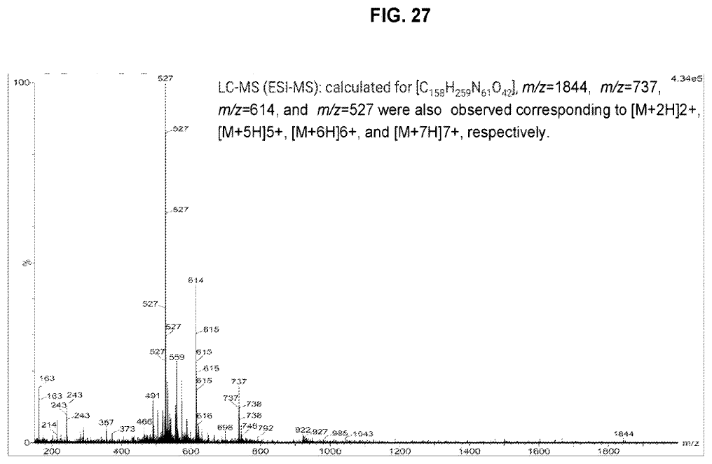

In this example, the linker that ties two PROTAC components enables the incapacitation mechanism. Normally this is a simple linker, such as a sequence of glycines, which tethers the two substrates. The substrates will bind the protein and ligase respectively, thus bringing them in sufficient proximity to each other so that the ligase can act on the protein. For the adaptive drug the recognition sequence is also added to this glycine linker. Without recognition of and reaction with the recognition sequence, the PROTAC components are connected with an intact linker, and are therefore in sufficient proximity to function. However when the recognition sequence is recognized and cleaved by the prostasin enzyme, the two pieces of the PROTAC drug (the protein substrate and the ligase substrate) are severed and separated from each other, thus dismembering the drug and incapacitating it. An example is shown in FIG. 26 where a purely peptide PROTAC for the tropomyosin receptor kinase A (TrkA) from the literature (Hines, J., Gough, J. D., Corson, T. W., & Crews, C. M. (2013)) is used for an adaptive drug with the dismemberment mode. The TrkA/PI3K pathway is known to promote cell survival and growth and thus is an established target for anti-cancer therapies. A PROTAC can starve cancers cells of this key protein but can also potentially starve healthy cells as well. Thus making the PROTAC adaptive through the use of the dismemberment incapacitation mechanism in the case of healthy cells has the potential to improve its selectivity and reduce off-target effects. FIG. 27 shows the characterization of the adaptive peptide PROTAC.

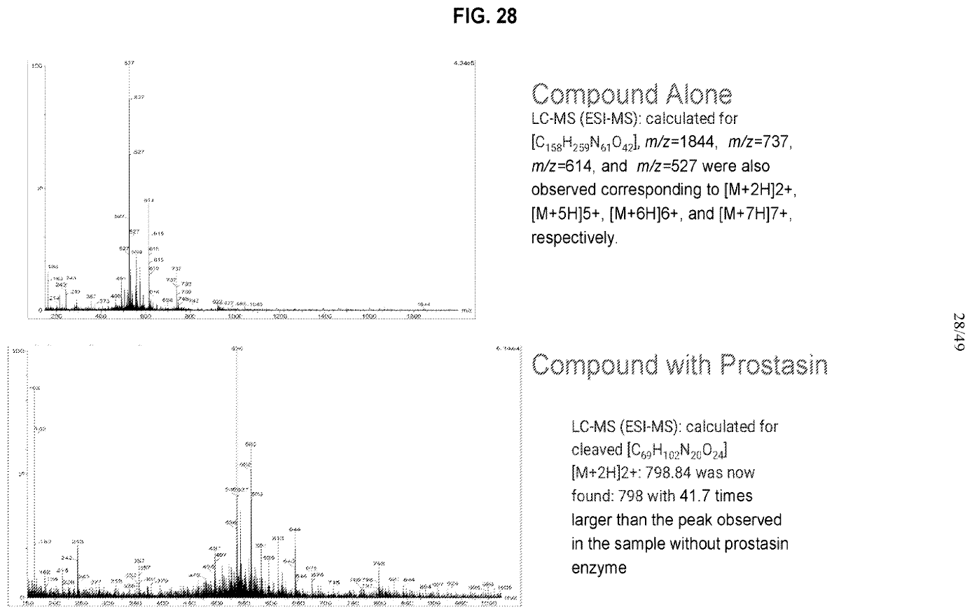

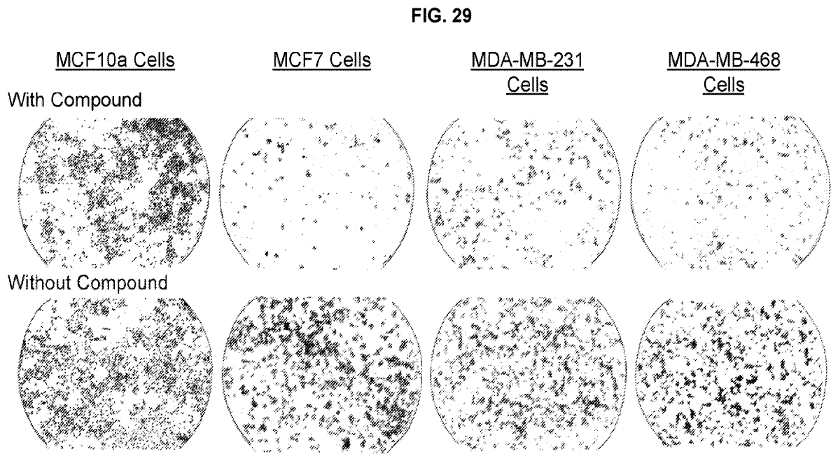

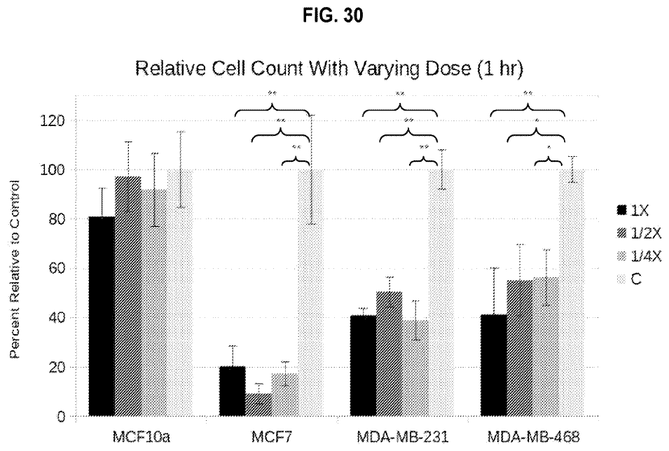

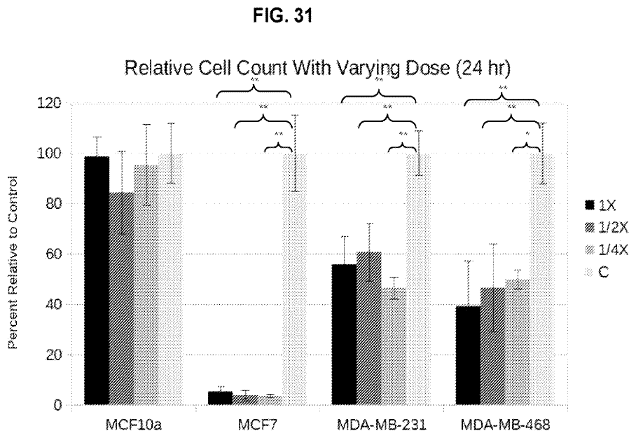

FIG. 29 then shows selective reduction in cell numbers for a number of cancer cell lines while sparing the normal cell line control. FIGS. 30 and 31 show the variation of this cell number reduction with applied dose and incubation time. Demonstration of activity of the drug when the incapacitation mechanism is not engaged can be done using a western blot for the TrkA protein from the cell lysate after drug application. Demonstration of the engagement of the incapacitation mechanism is shown by incubation of the adaptive peptide PROTAC with the prostasin enzyme revealing the dismembered TrkA ligand fraction in LC/MS analysis shown in FIG. 28. The strategy of this example can be generalized for any peptide based PROTAC for TrkA or any other target.

### Small Molecule PROTAC Drug Active

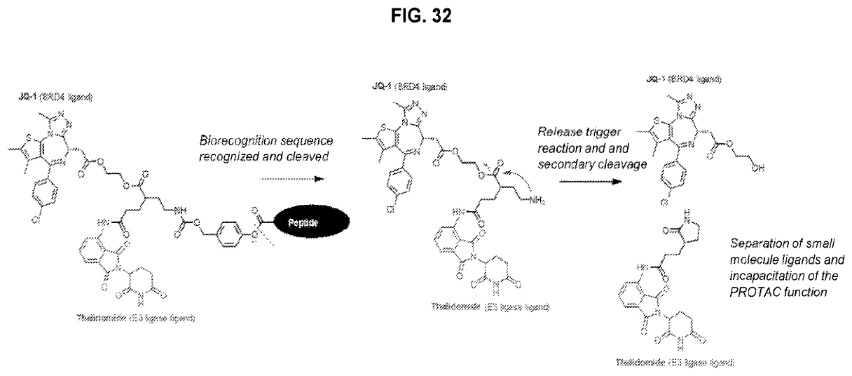

The epigenetic profile of a cell is maintained by three broad classes of enzymes-known as readers, writers and erasers-which affect the chemical and physical structure of DNA to express or repress different sets of genes. This is critical to the regulation of gene expression and as such their aberrant functionality has been linked to more dysregulated states, like cancer, autoimmune diseases and neurodegeneration. Thus there is a large market to inhibit these aberrant enzymes. One target can be the BET bromodomain. A PROTAC describe in the literature (Wang, C., Zhang, Y., Yang, S., Chen, W., & Xing, D. (2022)) builds of an inhibitor for the protein BET bromodomain family called (+)-JQ-1 and furthers its effect by using it as substrate to degrade the protein entirely. Of course these BET bromodomain proteins are also necessary and present in healthy cells, so selective effects of this BET bromodomain PROTAC to, for instance, cancer cells would help reduce side and off-target effects. FIG. 32 shows the design of such an adaptive PROTAC which incorporates the release trigger architecture.

Demonstration of activity of the drug when the incapacitation mechanism is not engaged can be done using a western blot for the BET bromodomain proteins like (BRD2, 3 or 4) from the cell lysate after drug application. Demonstration of the engagement of the incapacitation mechanism can be done by incubation with the prostasin enzyme and analysis of the byproducts (in this case the separated ligands for (+)-JQ-1 and the E3 ligase). In addition the same cell number assays can be done as before, varying dose and incubation time.

### Example 7. Mode 4 Increased Clearability

When the mode of increased clearability is engaged, the incapacitation mechanism alters some property of the drug to increase the likelihood of it being cleared from the cell, tissue, organ or the body entirely before it can reach its target, thereby incapacitating it in its otherwise intended biological effect.

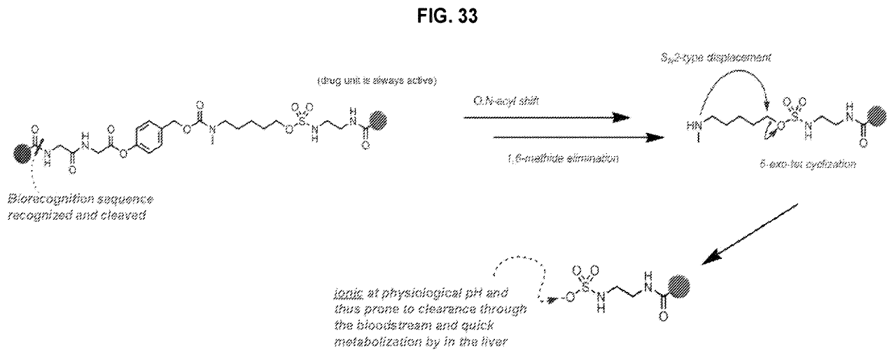

Small molecules often effectively disperse throughout the tissue, and thus they need to be cleared when and where not needed. The modality of increased clearability would offer a route to make such drug actives more precise in their utilization, with less off target and side effects. This is done as the incapacitation mechanism adaptively adds/reveals a functional group tag on the drug active that marks it for clearance from the body. In one demonstration of this in FIG. 33, the functional group tag is an anionic sulfonate.

### Metabolic Pathway Inhibitors

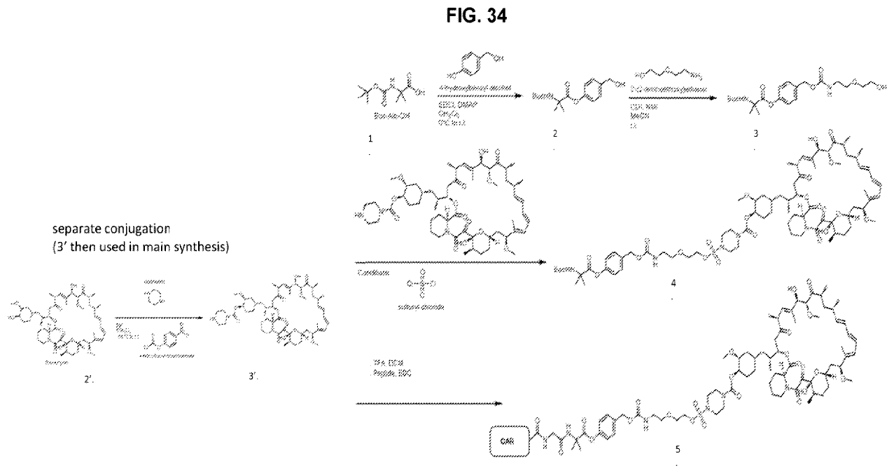

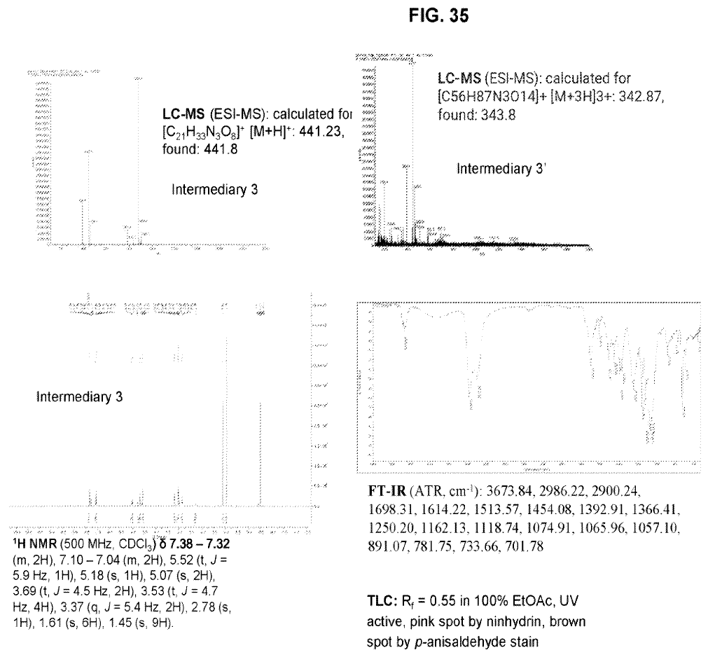

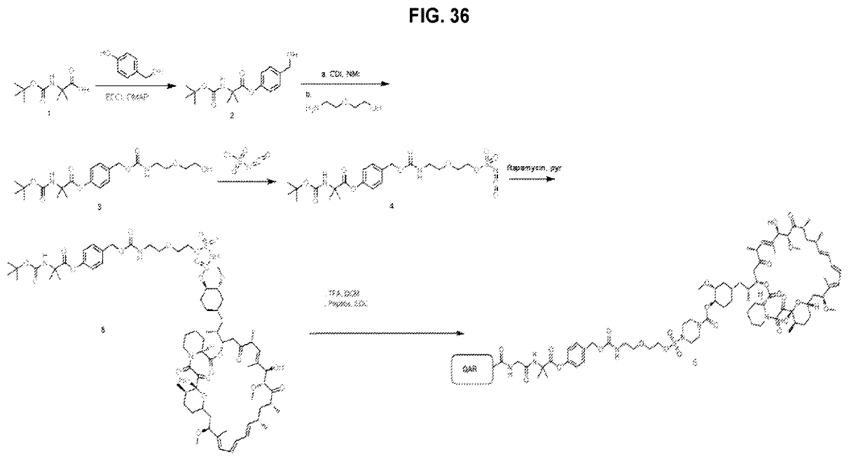

Cells employ certain key pathways to regulate nutrient metabolism for example the mTOR, AKT and PI3K pathways. Inhibitors of these pathways like the drug rapamycin can promote a more nutrient starved state, which has been shown to have a number of benefits in slowing down abnormal growth (like in cancers) and upregulating pro-longevity pathways and behaviors like increasing autophagy or mitochondrial turnover. However, too much nutrient starvation could potentially also lead to cell and tissue wasting. Thus, FIG. 34 shows a design to adaptively incapacitate rapamycin through the increased clearance modality. FIG. 35 shows characterization of intermediaries in the synthesis. FIG. 36 shows an alternative design for the adaptively rapamycin.

The drug could be tested in similar ways as the other compounds. This includes incubation with the prostasin enzyme and analysis of the byproducts with LC/MS (in this case the sulfonated rapamycin) to demonstrate engagement of the incapacitation mechanism. Additionally, cell survival and growth assessment can be done to demonstrate selective inhibition leading to death/slowed growth in the cancer cells while not significantly affecting the healthy cells. Demonstration of activity of the drug when the incapacitation mechanism is not engaged can be assessed by western blot on lysates of cells treated with the adaptive rapamycin for phosphorylated S6 S240/S244 (mTORC1) and AKT S473 (mTORC2) as described in the literature (Schreiber, K. H., Arriola Apelo, S. I., Yu, D., Brinkman, J. A., Velarde, M. C., Syed, F. A., . . . & Lamming, D. W. (2019)).

### Epigenetic Pathway Inhibitors

As mentioned in the drug active dismemberment modality section, JQ-1 is a small molecule that binds to the epigenetic regulator family of BET bromodomain proteins. Instead of using it as the ligand for a PROTAC, FIG. 37 shows a design to adaptively incapacitate its basic function as a small molecule inhibitor through the increased clearance modality. An alternative strategy using phosphorylation instead of sulfonation is also depicted.

The drug could be tested in similar ways as the other compounds. This includes incubation with the prostasin enzyme and analysis of the byproducts with LC/MS (in this case the sulfonated or phospholyated JQ-1) to demonstrate engagement of the incapacitation mechanism. Additionally, cell survival and growth assessment can be done to demonstrate selective inhibition leading to death/slowed growth in the cancer cells while not significantly affecting the healthy cells, albeit with some simulation of clearance in the body. Demonstration of activity of the drug when the incapacitation mechanism is not engaged can be done with FRET based binding assays for BRD4 bromodomain 1 like the ones offered by Cayman Chemicals (Cat 600520).

### Example 8. Mode 5 with Cell Entry Limitation of a Large Active (Intercalating Agent)

When the mode of mobility deduction is engaged, mobility of the drug is halted or reduced in terms of reaching its target, thereby incapacitating it in its otherwise intended biological effect.

Intercalation in between the nucleotide base pairs can interfere with a DNA replication, which can further lead to cell apoptosis. Thus intercalating agents can be used in chemotherapy for slowing or clearing cancerous cell growth but may also, undesirably, do the same to the healthy cells. Positioning is critical for this type of drug to affect the DNA. Thus, the mobility reduction modality for the incapacitation mechanism can be used to prevent an intercalating agent from reaching its DNA target.

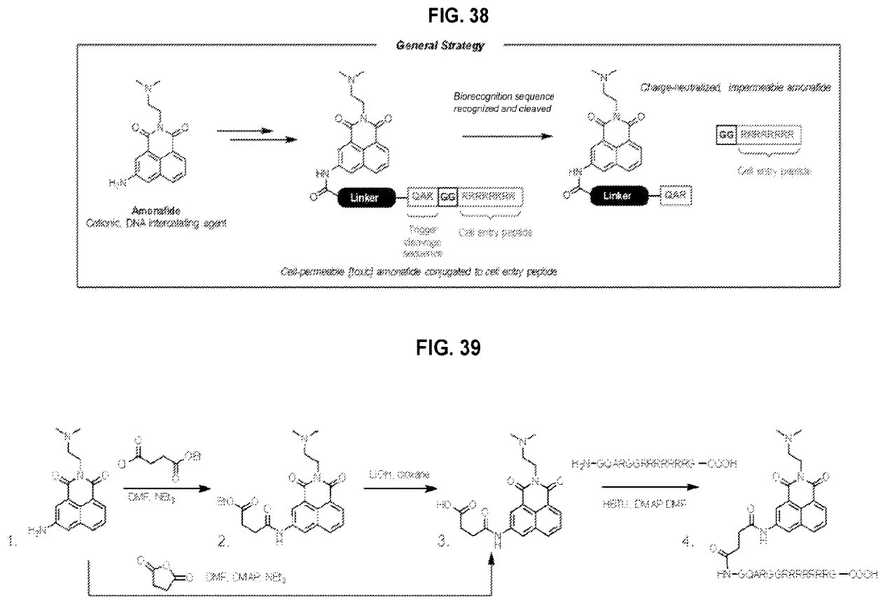

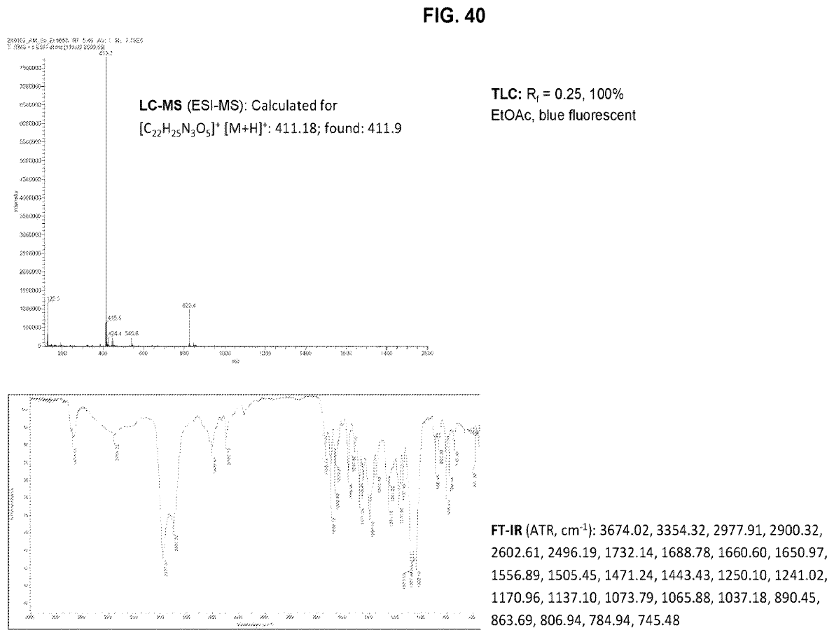

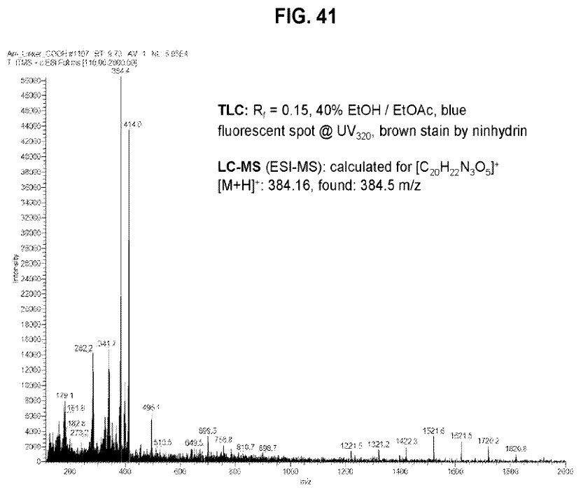

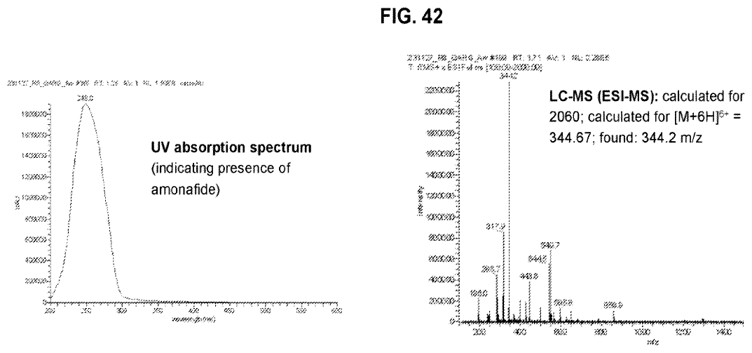

In this example, the adaptive intercalating agent can adaptively remove a cell entry element (specifically a cell entry peptide that enables passage through the cell membrane). The intercalating agent in the example is amonafide which further relies on a strongly cationic character to move it towards, bind to and intercalate within anionic polynucleotides of the DNA. Hence the highly cationic cell entry peptide adds another mobility function near the target site as well. In this example, the incapacitation mechanism involves the removal of the cell entry element for mobility reduction by adaptively controlling the linkage of the two. This is depicted in FIG. 38 where the two are adaptively linked through the biorecognition element and an additional linker piece. When the biorecognition element is recognized the cleavage occurs in a way to both remove the cell entry element but also reduce the charge on the remaining amonafide drug with the elements still affixed to it, decreasing its capacity to move through cell membrane and intercalate into DNA. The synthesis route is depicted in FIG. 39. The methods and characterization of key intermediates in the synthesis are shown in FIGS. 40-42. The strategy of this example can be generalized for a drug that requires a cell entry peptide to take effect.

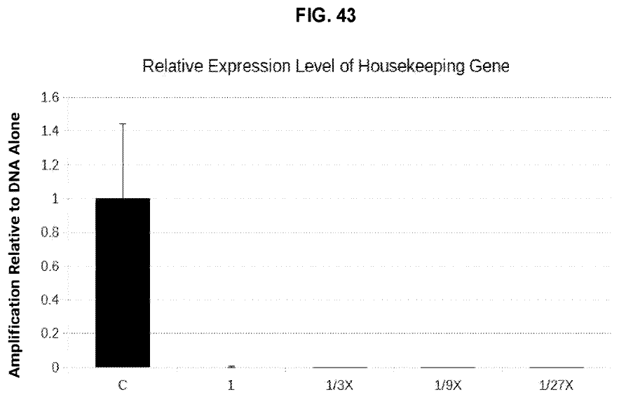

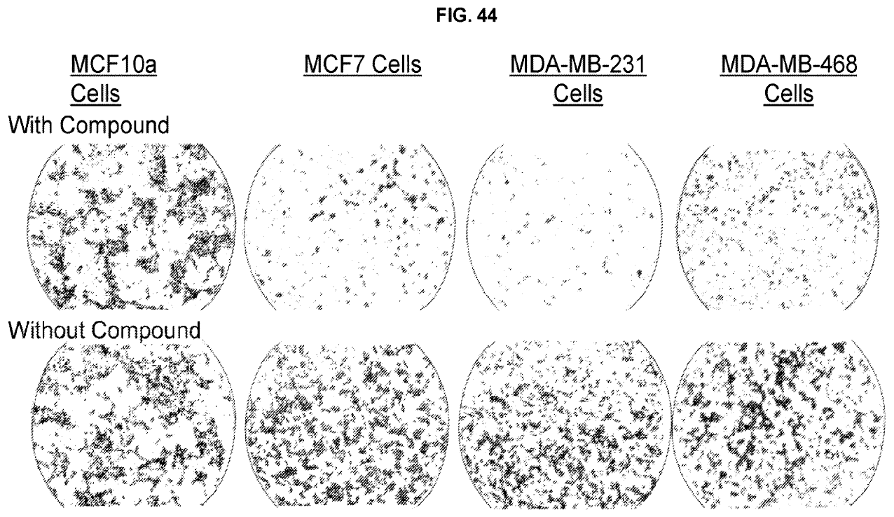

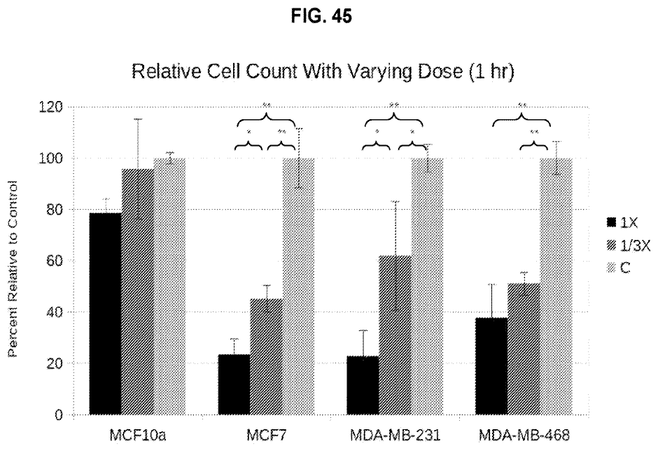

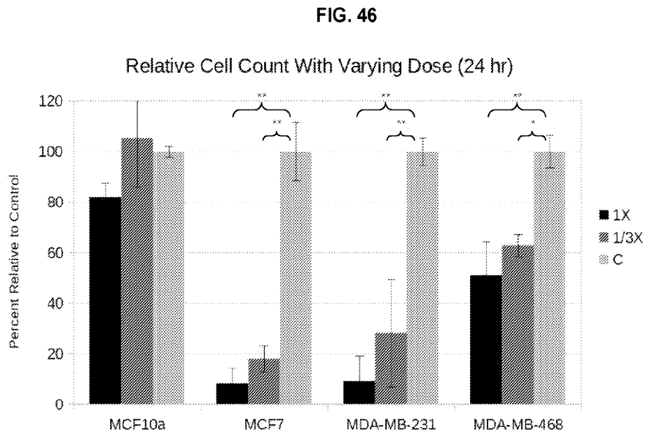

Demonstration of the activity of the adaptive drug is shown by qPCR in FIG. 43, where the drug prevents the replication of isolated cDNA. FIG. 44 shows selective reduction in cell numbers for a number of cancer cell lines while sparing the normal cell line control. FIGS. 45 and 46 then show the variation of this cell number reduction with applied dose and incubation time. Demonstration of the engagement of the incapacitation mechanism can be done by incubation with the prostasin enzyme and analysis of the byproducts with LC/MS (in this case the amonafide with the linker fragment and biorecognition element and the separated cell entry peptide).

The drug actives shown cover a number of different functions including inhibitors, activators, cytotoxins and PROTACs. They also cover different molecules, like small molecules, peptides, and conjugates of the two. Yet the concept of such adaptive drugs and the modalities of inactivation are not limited to such and extend on other types of function—like sequestration, biosynthesis, anabolic activity, or catabolic activity—and molecules—like nucleic acids, hormones, lipids, proteins, metabolites and other types of large molecules.

This adaptive drug architecture can also be combined with other auxiliary drug product elements. For instance, traditional targeting elements, release elements, localizing elements and encapsulation elements which aren't part of the drug's adaptive mechanism may be further combined with the compounds.

### Example 9. Mode 2 with Cofactor Dependent Drug

Certain biological processes target one biological molecule but require another biological molecule as a cofactor to execute. This is known, for instance in the function of many enzymes which require organic and sometimes metallic cofactors. In a similar sense, some drugs are also designed to utilize elements in situ other than the target to actually have a biological effect on the target. Thus in this example, the competitive disabling is implemented through the engagement of a competitor, not for the biological target or the intended activity, but for the cofactor that enables the drug to function.

FIG. 47 depicts such a drug in the inhibitor Bobcat 339 of the ten-eleven translocation methylcytosine dioxygenase 1 (TET1) and TET2. This small molecule needs to sequester free copper ions as a cofactor in order to function as an inhibitor. In this example depicted the adaptive drug has an acylated hydroxybipyridyl ligand as part of the incapacitation mechanism which will be released upon recognition and reaction of the biorecognition sequence. It will subsequently bind to free copper ions in the environment, thereby prohibiting Bobcat339 from using them as a cofactor and incapacitating the drug.

The drug could be tested in similar ways as the other compounds. This includes incubation with the prostasin enzyme and analysis of the byproducts with LC/MS (in this case the separate Bobcat339 and hydroxybipyridyl ligand) to demonstrate engagement of the incapacitation mechanism. Additionally, cell survival and growth assessment can be done to demonstrate selective inhibition leading to death/slowed growth in the cancer cells while not significantly affecting the healthy cells. Demonstration of activity of the drug when the incapacitation mechanism is not engaged can be done using a quantitative HPLC-ESI-MS/MS assay of TET activity as described in the literature (Weirath, N. A., Hurben, A. K., Chao, C., Pujari, S. S., Cheng, T., Liu, S., & Tretyakova, N. Y. (2022)).

### Example 10. Mode 3 with Hydroxy Containing Drug Active

This example of the drug active dismemberment modality involves adaptively separating and displacing a primary hydroxyl group from its functional location on a drug, thereby incapacitating it in its intended biological function. Hydroxyl groups are a common generic element of many drugs and FIG. 48 depicts an example for the discussed drug JQ-1.

The drug could be tested in similar ways as the other compounds. This includes incubation with the prostasin enzyme and analysis of the byproducts with LC/MS (dismembered JQ-1) to demonstrate engagement of the incapacitation mechanism. Additionally, cell survival and growth assessment can be done to demonstrate selective inhibition leading to death/slowed growth in the cancer cells while not significantly affecting the healthy cells. Demonstration of activity of the drug when the incapacitation mechanism is not engaged can be done using a quantitative HPLC-ESI-MS/MS assay of TET activity as described in the literature (Weirath, N. A., Hurben, A. K., Chao, C., Pujari, S. S., Cheng, T., Liu, S., & Tretyakova, N. Y. (2022)).

### Example 11. Mode 5 Immobilization of a Drug Active

This example demonstrates complete mobility reduction through adaptable affixation of a carbene to the drug molecule after the incapacitation mechanism is engaged, again with the release trigger architecture. This carbene can then react with environmental molecules to completely immobilize and incapacitate the drug active. FIG. 49 depicts an example for the discussed drug JQ-1.

The drug could be tested in similar ways as the other compounds. This includes incubation with the prostasin enzyme and analysis of the byproducts with LC/MS (in this case the carbene affixed JQ-1) to demonstrate engagement of the incapacitation mechanism. Additionally, cell survival and growth assessment can be done to demonstrate selective inhibition leading to death/slowed growth in the cancer cells while not significantly affecting the healthy cells. Demonstration of activity of the drug when the incapacitation mechanism is not engaged can be done with FRET based binding assays for BRD4 bromodomain 1 like the ones offered by Cayman Chemicals (Cat 600520).

From the foregoing, it will be appreciated that specific embodiments of the invention have been described herein for purposes of illustration, but that various modifications may be made without deviating from the scope of the invention. Accordingly, the invention is not limited by the appended claims.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A compound selected from the group consisting of:
C-terminus-LFLGARGGRRRPPP, IENPQYFSDAGQARGGALAPYIPRRRRRRRR. 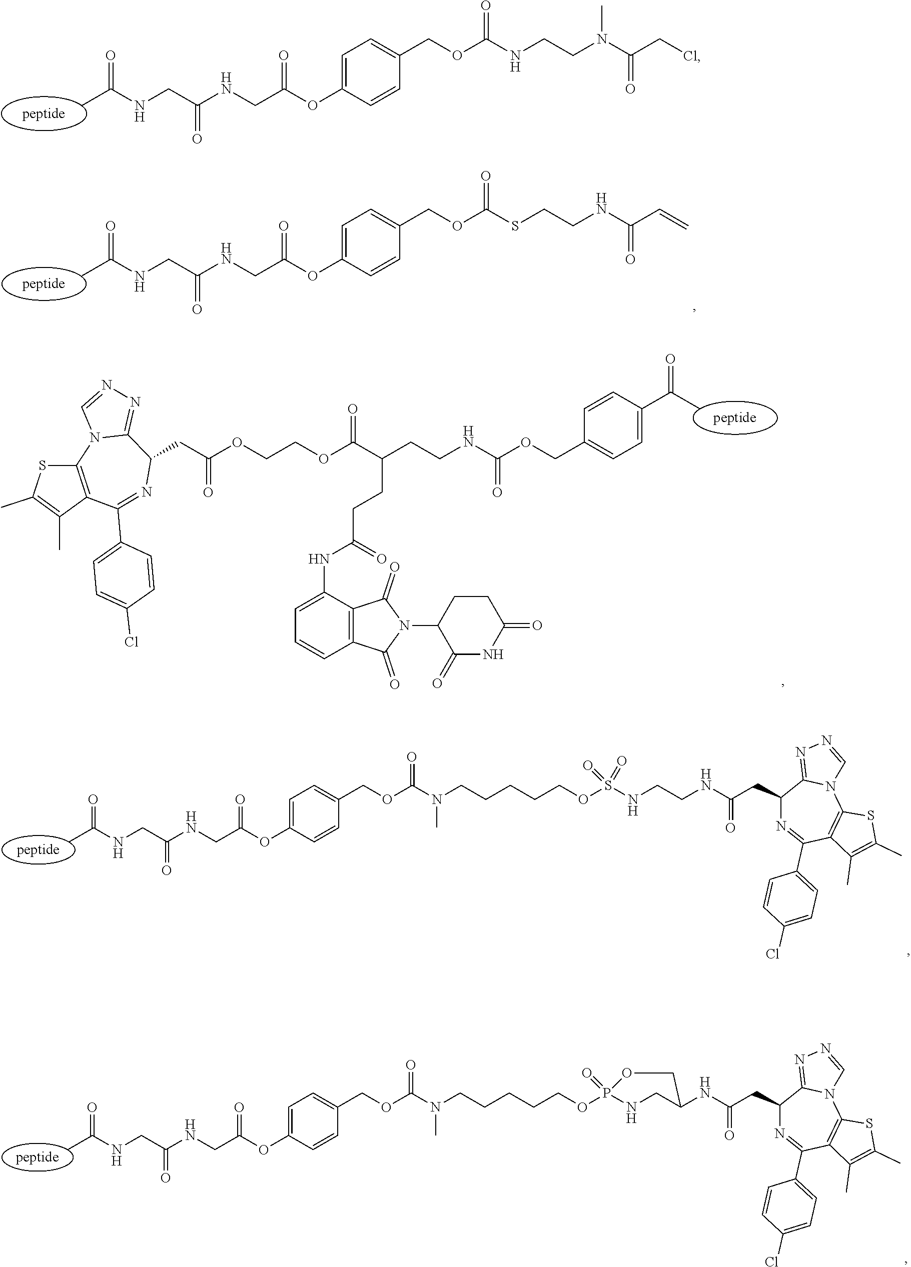 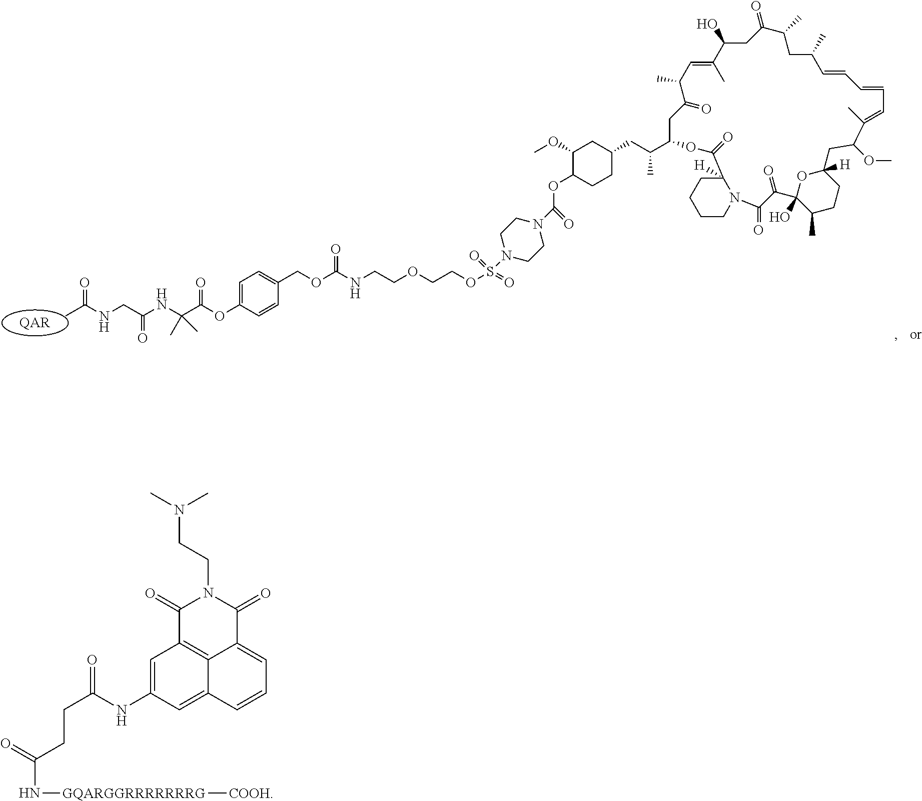
wherein the compound comprises:
(a) a biorecognition component comprising a substrate recognized by an enzyme,
(b) an incapacitation component, covalently linked to the biorecognition component, and
(c) a drug component covalently linked to the incapacitation component;
wherein the incapacitation component provides an incapacitation mechanism to incapacitate the drug component upon substrate recognition and reaction with the enzyme; and
wherein the drug is capable to execute its biological effect, but incapacitated after the recognition and reaction.

2-4. (canceled)

5. The compound of claim 1, wherein the enzyme cleaves or alters a covalent bond tethering the biorecognition component to engage the incapacitation mechanism.

6. The compound of claim 5, wherein the enzyme cleaves the covalent bond.

7. The compound of claim 6, wherein the covalent bond is an amide bond.

8. The compound of claim 1, wherein the enzyme is a hydrolase, an oxidoreductase, a transferase, a translocase, a lyase, a ligase, or an isomerase.

9. The compound of claim 8, wherein the hydrolase is a protease, a nuclease, a lipase, a phosphatase, or an esterase.

10. The compound of claim 9, wherein the protease is prostasin, matriptase, or CYLD lysin 63 deubiquitinase.

11. The compound of claim 9, wherein the nuclease is an endonuclease, an exonuclease, a DNase, a RNase, a topoisomerase, a recombinase, a ribozyme, or a RNA splicing enzyme.

12. The compound of claim 9, wherein the lipase is bile salt-dependent lipase, pancreatic lipase, lysosomal lipase, hepatic lipase, lipoprotein lipase, hormone-sensitive lipase, gastric lipase, endothelial lipase, or lingual lipase.

13. The compound of claim 9, wherein the phosphatase is phosphatidylinositol-3,4,5-trisphosphate 3-phosphatase, phosphatidic acid phosphatase type 2B, or inositol polyphosphate-5-phosphatase.

14. The compound of claim 9, wherein the esterase is acetylesterase or phosphodiesterase 2.

15. The compound of claim 1, wherein the enzyme alters the covalent bond between the biorecognition component and the incapacitation component by transfer of electrons or bonds from one substrate to another.

16. The compound of claim 15, wherein the enzyme is oxidoreductase or transferase.

17. The compound of claim 16, wherein the oxidoreductase is aldehyde dehydrogenase 2.

18. The compound of claim 16, wherein the transferase is methyltransferase or 3-hydroxy-3-methylglutaryl-CoA synthase 2.

19. The compound of claim 1, wherein the enzyme is located in a subject's extracellular space, cell membrane, cytoplasm, nucleus, or nuclear membrane.

20. The compound of claim 1, wherein the substrate comprises a peptide sequence or a biorecognition element, wherein the enzyme recognizes the peptide sequence or the biorecognition element.

21. The compound of claim 20, wherein the peptide sequence is recognized by protease.

22. The compound of claim 21, wherein the peptide sequence is QAR.

23. (canceled)

24. The compound of claim 1, wherein the incapacitation component is a set of linkers that comprises a release trigger covalently linked to the biorecognition element, wherein the release trigger is cleaved from the biorecognition element upon substrate biorecognition and reaction with the enzyme.

25-137. (canceled)


SEQUENCE LISTING (37 sequences)

SEQ ID NO: 1 - protein, 10 aa, synthetic construct
IENPQYFSDA

SEQ ID NO: 2 - protein, 14 aa, synthetic construct
LFLGARGGRRRPPP

SEQ ID NO: 3 - protein, 31 aa, synthetic construct
IENPQYFSDAGQARGGALAPYIPRRRRRRRR

SEQ ID NO: 4 - protein, 14 aa, synthetic construct
GQARGGRRRRRRRG

SEQ ID NO: 5 - protein, 7 aa, synthetic construct
RRRRPPP

SEQ ID NO: 6 - protein, 6 aa, synthetic construct
GQARGG

SEQ ID NO: 7 - protein, 16 aa, synthetic construct
LFLGQARGGRRRRPPP

SEQ ID NO: 8 - protein, 7 aa, synthetic construct
ALAPYIP

SEQ ID NO: 9 - protein, 4 aa, synthetic construct
GQAR

SEQ ID NO: 10 - protein, 8 aa, synthetic construct
RRRRRRRR

SEQ ID NO: 11 - protein, 14 aa, synthetic construct
GQARGGRRRRRRRG

SEQ ID NO: 12 - protein, 4 aa, synthetic construct
KHYR

SEQ ID NO: 13 - protein, 4 aa, synthetic construct
PRLR

SEQ ID NO: 14 - protein, 4 aa, synthetic construct
QARG

SEQ ID NO: 15 - protein, 15 aa, synthetic construct
RRRRNRTRRNRRRVR

SEQ ID NO: 16 - protein, 12 aa, synthetic construct
RRRRRRRRRRRR

SEQ ID NO: 17 - protein, 17 aa, synthetic construct
PPPPPPPPPRRRRRRRW

SEQ ID NO: 18 - protein, 13 aa, synthetic construct
GRKKRRQRRRPPQ

SEQ ID NO: 19 - protein, 16 aa, synthetic construct
RQIKIWFQNRRMKWKK

SEQ ID NO: 20 - protein, 9 aa, synthetic construct
RRRRRRRRR

SEQ ID NO: 21 - protein, 9 aa, synthetic construct
RRARRPRGR

SEQ ID NO: 22 - protein, 6 aa, synthetic construct
PKLKRQ

SEQ ID NO: 23 - protein, 4 aa, synthetic construct
RPRK

SEQ ID NO: 24 - protein, 21 aa, synthetic construct
GKRKLITSEEERSPAKRGRKS

SEQ ID NO: 25 - protein, 22 aa, synthetic construct
KGKKGRTQKEKKAARARSKGKN

SEQ ID NO: 26 - protein, 28 aa, synthetic construct
RKRCAAGVGGGPAGCPAPGSTPLKKPRR

SEQ ID NO: 27 - protein, 36 aa, synthetic construct
RKPVTAQERQREREEKRRRRQERAKEREKRRQERER

SEQ ID NO: 28 - protein, 27 aa, synthetic construct
RSGGNHRRNGRGGRGGYNRRNNGYHPY

SEQ ID NO: 29 - protein, 27 aa, synthetic construct
TLLLRETMNNLGVSDHAVLSRKTPQPY

SEQ ID NO: 30 - protein, 18 aa, synthetic construct
PGKMDKGEHRQERRDRPY

SEQ ID NO: 31 - protein, 12 aa, synthetic construct
RRARRPRGRQAR

SEQ ID NO: 32 - protein, 31 aa, synthetic construct; identical to SEQ ID NO: 3

SEQ ID NO: 33 - protein, 14 aa, synthetic construct
IENPQYFSDAGQAR

SEQ ID NO: 34 - protein, 17 aa, synthetic construct
GGALAPYIPRRRRRRRR

SEQ ID NO: 35 - protein, 13 aa, synthetic construct
QARGGRRRRRRRR

SEQ ID NO: 36 - protein, 10 aa, synthetic construct
GGRRRRRRRR

SEQ ID NO: 37 - protein, 4 aa, synthetic construct
QARG